(19) United States
(12) Patent Application Publication
(10) Pub. No.: US 2007/0264279 A1
(43) Pub. Date: Nov. 15, 2007

(54) Compositions and methods comprising a MAGE-b antigen

(76) Inventors: Claudia Gravekamp, San Francisco, CA (US); Yvonne Paterson, Philadelphia, PA (US); Paulo Maciag, Princeton, NJ (US)

Correspondence Address: PEARL COHEN ZEDEK LATZER, LLP, 1500 BROADWAY 12TH FLOOR, NEW YORK, NY, 10036, US

(21) Appl. No.: 11/727,889

(22) Filed: Mar. 28, 2007

Related U.S. Application Data
(63) Continuation-in-part of application No. 11/223,945, filed on Sep. 13, 2005.
Application No. 11/223,945 is a continuation-in-part of application No. 10/949,667, filed on Sep. 24, 2004.
Application No. 10/949,667 is a continuation-in-part of application No. 10/441,851, filed on May 20, 2003, now Pat. No. 7,135,188.
Application No. 10/441,851 is a continuation-in-part of application No. 09/535,212, filed on Mar. 27, 2000, now Pat. No. 6,565,852.
Application No. 09/535,212 is a continuation-in-part of application No. 08/336,372, filed on Nov. 8, 1994, now Pat. No. 6,051,237.

(51) Int. Cl. A61K 39/02; A61P 35/04; C07H 21/04; C07K 14/195; C12N 1/20; C12N 15/63

(52) U.S. Cl. 424/190.1; 424/200.1; 435/252.3; 435/320.1; 530/350; 536/23.7

(57) ABSTRACT

The present invention provides MAGE-b peptides, recombinant polypeptides comprising same, recombinant nucleotide molecules encoding same, recombinant Listeria strains comprising same, and immunogenic and therapeutic methods utilizing same.

### CROSS REFERENCE TO RELATED APPLICATIONS

This application is a Continuation-in-Part of co-pending U.S. application Ser. No. 11/223,945, filed Sep. 13, 2005, which is a Continuation-in-Part of co-pending U.S. application Ser. No. 10/949,667, filed Sep. 24, 2004, which is a Continuation-in-Part of co-pending U.S. application Ser. No. 10/441,851, filed May 20, 2003, now U.S. Pat. No. 7,135,188, which is a Continuation-in-Part of U.S. application Ser. No. 09/535,212, filed Mar. 27, 2000, now U.S. Pat. No. 6,767,542, which is a Continuation-in-Part of U.S. application Ser. No. 08/336,372, filed Nov. 8, 1994, now U.S. Pat. No. 6,051,237. These applications are hereby incorporated in their entirety by reference herein.

### STATEMENT REGARDING FEDERALLY SPONSORED RESEARCH OR DEVELOPMENT

The invention described herein was supported in whole or in part by grants from The National Institutes of Health (Grant No. 1ROAG023096-01). The government has certain rights in the invention

### FIELD OF THE INVENTION

The present invention provides MAGE-b peptides, recombinant polypeptides comprising same, recombinant nucleotide molecules encoding same, recombinant Listeria strains comprising same, and immunogenic and therapeutic methods utilizing same.

### BACKGROUND OF THE INVENTION

Stimulation of an immune response is dependent upon the presence of antigens recognized as foreign by the host immune system. Bacterial antigens such as Salmonella enterica and Mycobacterium bovis BCG remain in the phagosome and stimulate CD4 T-cells via antigen presentation through major histocompatibility class II molecules. In contrast, bacterial antigens such as Listeria monocytogenes exit the phagosome into the cytoplasm. The phagolysosomal escape of L. monocytogenes is a unique mechanism which facilitates major histocompatibility class I antigen presentation of listerial antigens. This escape is dependent upon the pore-forming sulfhydryl-activated cytolysin, listeriolysin O (LLO).

ActA is a surface-associated Listerial protein, and acts as a scaffold in infected host cells to facilitate the polymerization, assembly and activation of host actin polymers in order to propel the Listeria organism through the cytoplasm. Shortly after entry into the mammalian cell cytosol, L. monocytogenes induces the polymerization of host actin filaments and uses the force generated by actin polymerization to move, first intracellularly and then from cell to cell. A single bacterial protein, ActA is responsible for mediating actin nucleation and actin-based motility. The ActA protein provides multiple binding sites for host cytoskeletal components, thereby acting as a scaffold to assemble the cellular actin polymerization machinery. The NH₂ terminus of ActA binds to monomeric actin and acts as a constitutively active nucleation promoting factor by stimulating the intrinsic actin nucleation activity. ActA and hly are both members of the 10-kb gene cluster regulated by the transcriptional activator PrfA, and is upregulated approximately 226-fold in the mammalian cytosol.

There exists a long-felt need to develop compositions and methods to enhance the immunogenicity of antigens, especially antigens useful in the prevention and treatment of tumors and intracellular pathogens.

### SUMMARY OF THE INVENTION

The present invention provides MAGE-b peptides, recombinant polypeptides comprising same, recombinant nucleotide molecules encoding same, recombinant Listeria strains comprising same, and immunogenic and therapeutic methods utilizing same.

In another embodiment, the present invention provides a recombinant Listeria strain expressing a MAGE-b peptide. In another embodiment, the sequence of the MAGE-b peptide comprises a sequence selected from SEQ ID No: 34-39. In another embodiment, the sequence of the MAGE-b peptide comprises the sequence of an immunogenic peptide fragment of a peptide represented by SEQ ID No: 34-39. In another embodiment, the recombinant Listeria strain expresses a recombinant polypeptide that comprises a MAGE-b peptide. In another embodiment, the recombinant Listeria strain comprises a recombinant polypeptide, wherein the recombinant peptide comprises a MAGE-b peptide. In another embodiment, the recombinant Listeria strain comprises a recombinant nucleotide encoding the recombinant polypeptide. Each possibility represents a separate embodiment of the present invention.

In another embodiment, the present invention provides a vaccine comprising a recombinant Listeria strain of the present invention and an adjuvant.

In another embodiment, the present invention provides an immunogenic composition comprising a recombinant Listeria strain of the present invention.

In another embodiment, the present invention provides a recombinant polypeptide, comprising a MAGE-b peptide operatively linked to a non-MAGE-b peptide. In another embodiment, the non-MAGE-b peptide is an LLO peptide. In another embodiment, the non-MAGE-b peptide is an ActA peptide. In another embodiment, the non-MAGE-b peptide is a PEST-like sequence peptide. In another embodiment, the non-MAGE-b peptide is any other type of peptide known in the art. Each possibility represents a separate embodiment of the present invention.

In another embodiment, the present invention provides a vaccine comprising a recombinant polypeptide of the present invention and an adjuvant.

In another embodiment, the present invention provides an immunogenic composition comprising a recombinant polypeptide of the present invention.

In another embodiment, the present invention provides a recombinant vaccine vector encoding a recombinant polypeptide of the present invention.

In another embodiment, the present invention provides a nucleotide molecule encoding a recombinant polypeptide of the present invention.

In another embodiment, the present invention provides a vaccine comprising a nucleotide molecule of the present invention and an adjuvant.

In another embodiment, the present invention provides an immunogenic composition comprising a nucleotide molecule of the present invention.

In another embodiment, the present invention provides a recombinant vaccine vector comprising a nucleotide molecule of the present invention.

In another embodiment, the present invention provides a recombinant polypeptide comprising a fragment of a MAGE-b protein, wherein the fragment consists of amino acids 105-220 of the MAGE-b protein

In another embodiment, the present invention provides a recombinant polypeptide comprising a fragment of a MAGE-b protein, wherein the fragment consists of AA 2-117 of the MAGE-b protein. In another embodiment, the present invention provides a recombinant polypeptide comprising a fragment of a MAGE-b protein, wherein the fragment consists of amino acids 204-330 of the MAGE-b protein. Each possibility represents a separate embodiment of the present invention.

In another embodiment, the present invention provides a recombinant Listeria strain comprising a recombinant polypeptide of the present invention.

In another embodiment, the present invention provides a method of inducing an anti-MAGE-b immune response in a subject, comprising administering to the subject a composition comprising a recombinant Listeria strain of the present invention, thereby inducing an anti-MAGE-b immune response in a subject.

In another embodiment, the present invention provides a method of treating a MAGE-b expressing tumor in a subject, the method comprising the step of administering to the subject a composition comprising a recombinant Listeria strain of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject a composition comprising a recombinant Listeria strain of the present invention, whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of inducing an anti-MAGE-b immune response in a subject, comprising administering to the subject an immunogenic composition comprising a recombinant polypeptide of the present invention, thereby inducing an anti-MAGE-b immune response in a subject.

In another embodiment, the present invention provides a method of treating a MAGE-b expressing tumor in a subject, the method comprising the step of administering to the subject an immunogenic composition comprising a recombinant polypeptide of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject an immunogenic composition comprising a recombinant polypeptide of the present invention, whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of inducing an anti-MAGE-b immune response in a subject, comprising administering to the subject an immunogenic composition comprising a nucleotide molecule of the present invention, thereby inducing an anti-MAGE-b immune response in a subject.

In another embodiment, the present invention provides a method of treating a MAGE-b expressing tumor in a subject, the method comprising the step of administering to the subject an immunogenic composition comprising a nucleotide molecule of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject an immunogenic composition comprising a nucleotide molecule of the present invention whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

### BRIEF DESCRIPTION OF THE DRAWINGS

FIG. 1. Lm-E7 and Lm-LLO-E7 use different expression systems to express and secrete E7. Lm-E7 was generated by introducing a gene cassette into the orfz domain of the L. monocytogenes genome (A). The hly promoter drives expression of the hly signal sequence and the first five amino acids (AA) of LLO followed by HPV-16 E7. B), Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. A. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles. B. Tumor immunotherapeutic efficacy of LLO-Ova fusions.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)—(no-TC-1 control).

FIG. 5. Tumor immunotherapeutic efficacy of NP antigen expressed in LM. Tumor size in millimeters in mice is shown at 10, 17, 24, and 38 days post tumor-inoculation. Naive mice: X's; mice administered Lm-LLO-NP: filled diamonds; Lm-NP: squares; Lm-Gag: open circles.

FIG. 6. Depiction of vaccinia virus constructs expressing different forms of HPV16 E7 protein.

FIG. 7. VacLLOE7 causes long-term regression of tumors established from 2×10⁵ TC-1 cells injected s.c. into C57BL/6 mice. Mice were injected 11 and 18 days after tumor challenge with 10⁷ PFU of VacLLOE7, VacSigE7LAMP-1, or VacE7/mouse i.p. or were left untreated (naive). 8 mice per treatment group were used, and the cross section for each tumor (average of 2 measurements) is shown for the indicated days after tumor inoculation.

FIG. 8. A. schematic representation of the plasmid inserts used to create 4 LM vaccines. Lm-LLO-E7 insert contains all of the Listeria genes used. It contains the hly promoter, the first 1.3 kb of the hly gene (which encodes the protein LLO), and the HPV-16 E7 gene. The first 1.3 kb of hly includes the signal sequence (ss) and the PEST region. Lm-PEST-E7 includes the hly promoter, the signal sequence, and PEST and E7 sequences but excludes the remainder of the truncated LLO gene. Lm-ΔPEST-E7 excludes the PEST region, but contains the hly promoter, the signal sequence, E7, and the remainder of the truncated LLO. Lm-E7epi has only the hly promoter, the signal sequence, and E7. B. Top panel: Listeria constructs containing PEST regions induce tumor regression. Bottom panel: Average tumor sizes at day 28 post-tumor challenge in 2 separate experiments. C. Listeria constructs containing PEST regions induce a higher percentage of E7-specific lymphocytes in the spleen. Average and SE of data from 3 experiments are depicted.

FIG. 9. Tumor size in mice administered Lm-ActA-E7 (rectangles), Lm-E7 (ovals), Lm-LLO-E7 (X), and naive mice (non-vaccinated; solid triangles).

FIG. 10. A. Induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive). B. Induction and penetration of E7 specific CD8⁺ cells in the spleens and tumors of the mice described for (A).

FIG. 11. Listeria constructs containing PEST regions induce a higher percentage of E7-specific lymphocytes within the tumor. A. representative data from 1 experiment. B. average and SE of data from all 3 experiments.

FIG. 12: Development and characterization of the Listeria-based construct. Left panel: Cloning of Mage-b fragments and complete Mage-b in Listeria vector as fusion protein with Listeriolysin O (LLO), under the control of the hemolysin promoter (Phly). Right panel: Western blotting of Mage-b proteins (encoded by Mage-b fragments or complete Mage-b; arrows) secreted by LM in culture medium. Anti-myc (top) and anti-pest (bottom) antibodies were used. Lane 1: Mage-b/1st; lane 2: Mage-b/2nd; lane 3: Mage-b/3rd; lane 4: Mage-b/complete.

FIG. 13. Frequency of metastases per mouse. BALB/c mice with 4T1 metastases were injected with LM-LLO-Mage-b/2nd, LM-LLO (control), or Saline (control). Each triangle represents one mouse.

FIG. 14. Mage-b-specific immune responses in the spleens of mice without (ab) or with tumors (cd). Spleen cells were restimulated with autologous bone marrow cells expressing Mage-b (left), or with 4T1 tumor cells, expressing Mage-b (right). The number of IFNγ-producing cells per 200,000 spleen cells, was measured by ELISPOT. Results were analyzed by Mann-Whitney Test. (a) LM-LLO-Mage-b/2^{nd} vs. Saline p=0.0087, and LM-LLO-Mage-b/2^{nd} vs. LM-LLO p=0.0026; (b) LM-LLO-Mage-b/2^{nd} vs. Saline p=0.0065, and LM-LLO-Mage-b/2^{nd} vs. LM-LLO p=0.1999; (c) LM-LLO-Mage-b/2^{nd} vs. Saline p<0.0001, and LM-LLO-Mage-b/2^{nd} vs. LM-LLO p<0.0001; and (d) LM-LLO-Mage-b/2^{nd} vs. Saline p=0.0332, and LM-LLO-Mage-b/2ndvs. LM-LLO p=0.0056).

### DETAILED DESCRIPTION OF THE INVENTION

The present invention provides MAGE-b peptides, recombinant polypeptides comprising same, recombinant nucleotide molecules encoding same, recombinant Listeria strains comprising same, and immunogenic and therapeutic methods utilizing same.

In another embodiment, the present invention provides a recombinant Listeria strain expressing a MAGE-b peptide. In another embodiment, the sequence of the MAGE-b peptide comprises a sequence selected from SEQ ID No: 34-39. In another embodiment, the sequence of the MAGE-b peptide comprises the sequence of an immunogenic peptide fragment of a peptide represented by SEQ ID No: 34-39. In another embodiment, the recombinant Listeria strain expresses a recombinant polypeptide that comprises a MAGE-b peptide. In another embodiment, the recombinant Listeria strain comprises a recombinant polypeptide, wherein the recombinant peptide comprises a MAGE-b peptide. In another embodiment, the recombinant Listeria strain comprises a recombinant nucleotide encoding the recombinant polypeptide. Each possibility represents a separate embodiment of the present invention.

The MAGE-b peptide expressed by the recombinant Listeria strain is, in another embodiment, in the form of a fusion peptide. In another embodiment, the fusion peptide further comprises a non-MAGE-b peptide. In another embodiment, the non-MAGE-b peptide enhances the immunogenicity of the MAGE-b peptide. Each possibility represents a separate embodiment of the present invention.

In another embodiment, the present invention provides a vaccine comprising a recombinant Listeria strain of the present invention and an adjuvant.

In another embodiment, the present invention provides an immunogenic composition comprising a recombinant Listeria strain of the present invention.

In another embodiment, the present invention provides a method of inducing an anti-MAGE-b immune response in a subject, comprising administering to the subject a composition comprising a recombinant Listeria strain of the present invention, thereby inducing an anti-MAGE-b immune response in a subject.

In another embodiment, the present invention provides a method of treating a MAGE-b expressing tumor in a subject, the method comprising the step of administering to the subject a composition comprising a recombinant Listeria strain of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject a composition comprising a recombinant Listeria strain of the present invention, whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a recombinant polypeptide, comprising a MAGE-b peptide operatively linked to a non-MAGE-b peptide. In another embodiment, the non-MAGE-b peptide is an LLO peptide. In another embodiment, the non-MAGE-b peptide is an ActA peptide. In another embodiment, the non-MAGE-b peptide is a PEST-like sequence peptide. In another embodiment, the non-MAGE-b peptide is any other type of peptide known in the art. Each possibility represents a separate embodiment of the present invention.

As provided herein, a recombinant Listeria strain expressing an LLO-MAGE-b fusion protects mice from tumors and elicits formation of antigen-specific CTL. Thus, both Listeria strains expressing MAGE-b and LLO-MAGE-b fusions are antigenic and efficacious in vaccination methods.

Further, as provided herein, Lm-LLO-E7 induces regression of established subcutaneous HPV-16 immortalized tumors from C57B1/6 mice (Example 1). Further, as provided herein, Lm-LLO-NP protects mice from RENCA-NP, a renal cell carcinoma (Example 3). Further, as provided herein, fusion of antigens to ActA and PEST-like sequences produces similar results. Thus, non-hemolytic LLO, ActA, and PEST-like sequences are all efficacious at enhancing the immunogenicity of MAGE-b peptides.

In another embodiment, a recombinant polypeptide of methods and compositions of the present invention is made by a process comprising the step of translation of a nucleotide molecule encoding the recombinant polypeptide. In another embodiment, a recombinant polypeptide of methods and compositions of the present invention is made by a process comprising the step of chemically conjugating a polypeptide comprising the MAGE-b peptide to a polypeptide comprising the non-MAGE-b peptide. Each possibility represents a separate embodiment of the present invention.

In another embodiment, the present invention provides a vaccine comprising a recombinant polypeptide of the present invention and an adjuvant.

In another embodiment, the present invention provides an immunogenic composition comprising a recombinant polypeptide of the present invention.

In another embodiment, the present invention provides a recombinant vaccine vector encoding a recombinant polypeptide of the present invention.

In another embodiment, the present invention provides a nucleotide molecule encoding a recombinant polypeptide of the present invention.

In another embodiment, the present invention provides a vaccine comprising a nucleotide molecule of the present invention and an adjuvant.

In another embodiment, the present invention provides an immunogenic composition comprising a nucleotide molecule of the present invention.

In another embodiment, the present invention provides a recombinant vaccine vector comprising a nucleotide molecule of the present invention.

In another embodiment, the present invention provides a recombinant polypeptide comprising a fragment of a MAGE-b protein, wherein the fragment consists of amino acids 105-220 of the MAGE-b protein

In another embodiment, the present invention provides a recombinant polypeptide comprising a fragment of a MAGE-b protein, wherein the fragment consists of amino acids 2-117 of the MAGE-b protein. In another embodiment, the present invention provides a recombinant polypeptide comprising a fragment of a MAGE-b protein, wherein the fragment consists of amino acids 204-330 of the MAGE-b protein. Each possibility represents a separate embodiment of the present invention.

In another embodiment, a recombinant polypeptide of methods and compositions of the present invention further comprises a non-MAGE-b peptide. In another embodiment, the non-MAGE-b peptide enhances the immunogenicity of the fragment. In another embodiment, the non-MAGE-b peptide is a non-hemolytic LLO peptide. In another embodiment, the non-MAGE-b peptide is an ActA peptide. In another embodiment, the non-MAGE-b peptide is a PEST-like sequence-containing peptide. In another embodiment, the non-MAGE-b peptide is any other non-MAGE-b peptide known in the art. Each possibility represents a separate embodiment of the present invention.

In another embodiment, the recombinant polypeptide is made by a process comprising the step of translation of a nucleotide molecule encoding the recombinant polypeptide. In another embodiment, the recombinant polypeptide is made by a process comprising the step of chemically conjugating a polypeptide comprising the MAGE-b peptide to a polypeptide comprising the non-MAGE-b peptide. Each possibility represents a separate embodiment of the present invention.

In another embodiment, the present invention provides a recombinant Listeria strain comprising a recombinant polypeptide of the present invention. In another embodiment, the present invention provides a recombinant Listeria strain comprising a recombinant nucleotide encoding a recombinant polypeptide of the present invention. In another embodiment, the Listeria vaccine strain is a strain of the species Listeria monocytogenes (LM). In another embodiment, the present invention provides a composition comprising the Listeria strain. In another embodiment, the present invention provides an immunogenic composition comprising the Listeria strain. Each possibility represents a separate embodiment of the present invention.

The Listeria-containing composition of methods and compositions of the present invention is, in another embodiment, an immunogenic composition. In another embodiment, the composition is inherently immunogenic by virtue of its comprising a Listeria strain of the present invention. Each possibility represents a separate embodiment of the present invention.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. Each possibility represents a separate embodiment of the present invention.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the MAGE-b peptide-containing recombinant peptide. In another embodiment, the Listeria strain carries a plasmid comprising the gene encoding the MAGE-b peptide-containing recombinant peptide. Methods for passaging a recombinant Listeria strain through an animal host are well known in the art, and are described, for example, in United States Patent Application No. 2006/0233835, which is incorporated herein by reference. In another embodiment, the passaging is performed by any other method known in the art. Each possibility represents a separate embodiment of the present invention.

In another embodiment, the recombinant Listeria strain utilized in methods of the present invention has been stored in a frozen cell bank. In another embodiment, the recombinant Listeria strain has been stored in a lyophilized cell bank. Each possibility represents a separate embodiment of the present invention.

In another embodiment, the cell bank of methods and compositions of the present invention is a master cell bank. In another embodiment, the cell bank is a working cell bank. In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art. Each possibility represents a separate embodiment of the present invention.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States. Each possibility represents a separate embodiment of the present invention.

In another embodiment, a recombinant Listeria strain utilized in methods of the present invention is from a batch of vaccine doses.

In another embodiment, a recombinant Listeria strain utilized in methods of the present invention is from a frozen stock produced by a method disclosed herein.

In another embodiment, a recombinant Listeria strain utilized in methods of the present invention is from a lyophilized stock produced by a method disclosed herein.

In another embodiment, a cell bank, frozen stock, or batch of vaccine doses of the present invention exhibits viability upon thawing of greater than 90%. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year. Each possibility represents a separate embodiment of the present invention.

In another embodiment, a cell bank, frozen stock, or batch of vaccine doses of the present invention is cryopreserved by a method that comprises growing a culture of the Listeria strain in a nutrient media, freezing the culture in a solution comprising glycerol, and storing the Listeria strain at below −20 degrees Celsius. In another embodiment, the temperature is about −70 degrees Celsius. In another embodiment, the temperature is about ⁻70-⁻80 degrees Celsius.

In another embodiment, a cell bank, frozen stock, or batch of vaccine doses of the present invention is cryopreserved by a method that comprises growing a culture of the Listeria strain in a defined media of the present invention (as described below), freezing the culture in a solution comprising glycerol, and storing the Listeria strain at below −20 degrees Celsius. In another embodiment, the temperature is about −70 degrees Celsius. In another embodiment, the temperature is about ⁻70-⁻80 degrees Celsius. In another embodiment, any defined microbiological media of the present invention may be used in this method. Each defined microbiological media represents a separate embodiment of the present invention.

In another embodiment of methods and compositions of the present invention, the culture (e.g. the culture of a Listeria vaccine strain that is used to produce a batch of Listeria vaccine doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase. Each possibility represents a separate embodiment of the present invention.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art. Each possibility represents a separate embodiment of the present invention.

In another embodiment, the nutrient media utilized for growing a culture of a Listeria strain is LB. In another embodiment, the nutrient media is TB. In another embodiment, the nutrient media is a defined media. In another embodiment, the nutrient media is a defined media of the present invention. In another embodiment, the nutrient media is any other type of nutrient media known in the art. Each possibility represents a separate embodiment of the present invention.

In another embodiment of methods and compositions of the present invention, the step of growing is performed with a shake flask. In another embodiment, the flask is a baffled shake flask. In another embodiment, the growing is performed with a batch fermenter. In another embodiment, the growing is performed with a stirred tank or flask. In another embodiment, the growing is performed with an airflit fermenter. In another embodiment, the growing is performed with a fed batch. In another embodiment, the growing is performed with a continuous cell reactor. In another embodiment, the growing is performed with an immobilized cell reactor. In another embodiment, the growing is performed with any other means of growing bacteria that is known in the art. Each possibility represents a separate embodiment of the present invention.

In another embodiment, a constant pH is maintained during growth of the culture (e.g. in a batch fermenter). In another embodiment, the pH is maintained at about 7.0. In another embodiment, the pH is about 6. In another embodiment, the pH is about 6.5. In another embodiment, the pH is about 7.5. In another embodiment, the pH is about 8. In another embodiment, the pH is 6.5-7.5. In another embodiment, the pH is 6-8. In another embodiment, the pH is 6-7. In another embodiment, the pH is 7-8. Each possibility represents a separate embodiment of the present invention.

In another embodiment, a constant temperature is maintained during growth of the culture. In another embodiment, the temperature is maintained at about 37° C. In another embodiment, the temperature is 37° C. In another embodiment, the temperature is 25° C. In another embodiment, the temperature is 27° C. In another embodiment, the temperature is 28° C. In another embodiment, the temperature is 30° C. In another embodiment, the temperature is 32° C. In another embodiment, the temperature is 34° C. In another embodiment, the temperature is 35° C. In another embodiment, the temperature is 36° C. In another embodiment, the temperature is 38° C. In another embodiment, the temperature is 39° C. Each possibility represents a separate embodiment of the present invention.

In another embodiment, a constant dissolved oxygen concentration is maintained during growth of the culture. In another embodiment, the dissolved oxygen concentration is maintained at 20% of saturation. In another embodiment, the concentration is 15% of saturation. In another embodiment, the concentration is 16% of saturation. In another embodiment, the concentration is 18% of saturation. In another embodiment, the concentration is 22% of saturation. In another embodiment, the concentration is 25% of saturation. In another embodiment, the concentration is 30% of saturation. In another embodiment, the concentration is 35% of saturation. In another embodiment, the concentration is 40% of saturation. In another embodiment, the concentration is 45% of saturation. In another embodiment, the concentration is 50% of saturation. In another embodiment, the concentration is 55% of saturation. In another embodiment, the concentration is 60% of saturation. In another embodiment, the concentration is 65% of saturation. In another embodiment, the concentration is 70% of saturation. In another embodiment, the concentration is 75% of saturation. In another embodiment, the concentration is 80% of saturation. In another embodiment, the concentration is 85% of saturation. In another embodiment, the concentration is 90% of saturation. In another embodiment, the concentration is 95% of saturation. In another embodiment, the concentration is 100% of saturation. In another embodiment, the concentration is near 100% of saturation. Each possibility represents a separate embodiment of the present invention.

In another embodiment of methods and compositions of the present invention, the Listeria culture is flash-frozen in liquid nitrogen, followed by storage at the final freezing temperature. In another embodiment, the culture is frozen in a more gradual manner; e.g. by placing in a vial of the culture in the final storage temperature. In another embodiment, the culture is frozen by any other method known in the art for freezing a bacterial culture. Each possibility represents a separate embodiment of the present invention.

In another embodiment of methods and compositions of the present invention, the storage temperature of the culture is between ⁻20 and ⁻80 degrees Celsius (OC). In another embodiment, the temperature is significantly below ⁻20° C. In another embodiment, the temperature is not warmer than ⁻70° C. In another embodiment, the temperature is ⁻70° C. In another embodiment, the temperature is about ⁻70° C. In another embodiment, the temperature is ⁻20° C. In another embodiment, the temperature is about ⁻20° C. In another embodiment, the temperature is ⁻30° C. In another embodiment, the temperature is ⁻40° C. In another embodiment, the temperature is ⁻50° C. In another embodiment, the temperature is ⁻60° C. In another embodiment, the temperature is ⁻80° C. In another embodiment, the temperature is ⁻30-⁻70° C. In another embodiment, the temperature is ⁻40-⁻70° C. In another embodiment, the temperature is ⁻50-⁻70° C. In another embodiment, the temperature is ⁻60-⁻70° C. In another embodiment, the temperature is ⁻30-⁻80° C. In another embodiment, the temperature is ⁻40-⁻80° C. In another embodiment, the temperature is ⁻50-⁻80° C. In another embodiment, the temperature is ⁻60-⁻80° C. In another embodiment, the temperature is ⁻70-⁻80° C. In another embodiment, the temperature is colder than ⁻70° C. In another embodiment, the temperature is colder than ⁻80° C. Each possibility represents a separate embodiment of the present invention.

Methods for lyophilization and cryopreservation of recombinant Listeria strains are well known to those skilled in the art. Each possibility represents a separate embodiment of the present invention.

In another embodiment, the present invention provides a method of inducing an anti-MAGE-b immune response in a subject, comprising administering to the subject an immunogenic composition comprising a recombinant polypeptide of the present invention, thereby inducing an anti-MAGE-b immune response in a subject.

In another embodiment, the present invention provides a method of treating a MAGE-b expressing tumor in a subject, the method comprising the step of administering to the subject an immunogenic composition comprising a recombinant polypeptide of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject an immunogenic composition comprising a recombinant polypeptide of the present invention, whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of inducing an anti-MAGE-b immune response in a subject, comprising administering to the subject an immunogenic composition comprising a nucleotide molecule of the present invention, thereby inducing an anti-MAGE-b immune response in a subject.

In another embodiment, the present invention provides a method of treating a MAGE-b expressing tumor in a subject, the method comprising the step of administering to the subject an immunogenic composition comprising a nucleotide molecule of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject an immunogenic composition comprising a nucleotide molecule of the present invention whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of inducing an anti-MAGE-b immune response in a subject, comprising administering to the subject a composition comprising a recombinant Listeria strain, wherein the strain comprises a recombinant polypeptide of the present invention, thereby inducing an anti-MAGE-b immune response in a subject.

In another embodiment, the present invention provides a method of treating a MAGE-b expressing tumor in a subject, the method comprising the step of administering to the subject a composition comprising a recombinant Listeria strain, wherein the strain comprises a recombinant polypeptide of the present invention, whereby the subject mounts an immune response against the MAGE-b expressing tumor, thereby treating a MAGE-b expressing tumor in a subject. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of protecting a human subject against a MAGE-b expressing tumor, the method comprising the step of administering to the human subject a composition comprising a recombinant Listeria strain, wherein the strain comprises a recombinant polypeptide of the present invention whereby the subject mounts an immune response against the MAGE-b expressing tumor, thereby protecting a human subject against a MAGE-b expressing tumor. In another embodiment, the MAGE-b expressing tumor is a MAGE-b expressing breast cancer. In another embodiment, the MAGE-b expressing tumor is a MAGE-b expressing breast carcinoma. Each possibility represents a separate embodiment of the present invention.

In another embodiment, the present invention provides a method of impeding a growth of a MAGE-b expressing breast cancer tumor in a subject, comprising administering to the subject a composition comprising a recombinant Listeria strain of the present invention, whereby the subject mounts an immune response against a pericyte of a vasculature of the solid tumor, thereby impeding a growth of a MAGE-b expressing breast cancer tumor in a subject.

In another embodiment, the present invention provides a method of overcoming an immune tolerance of a subject to a MAGE-b expressing breast cancer tumor, comprising administering to the subject a composition comprising a recombinant Listeria strain of the present invention, whereby the subject mounts an immune response against a pericyte of a vasculature of the solid tumor, thereby overcoming an immune tolerance of a subject to a MAGE-b expressing breast cancer tumor.

In another embodiment, the present invention provides a method of impeding a growth of a MAGE-b expressing breast cancer tumor in a subject, comprising administering to the subject an immunogenic composition comprising a recombinant polypeptide of the present invention, whereby the subject mounts an immune response against a pericyte of a vasculature of the solid tumor, thereby impeding a growth of a MAGE-b expressing breast cancer tumor in a subject.

In another embodiment, the present invention provides a method of overcoming an immune tolerance of a subject to a MAGE-b expressing breast cancer tumor, comprising administering to the subject an immunogenic composition comprising a recombinant polypeptide of the present invention, whereby the subject mounts an immune response against a pericyte of a vasculature of the solid tumor, thereby overcoming an immune tolerance of a subject to a MAGE-b expressing breast cancer tumor.

In another embodiment, the present invention provides a method of impeding a growth of a MAGE-b expressing breast cancer tumor in a subject, comprising administering to the subject an immunogenic composition comprising a nucleotide molecule of the present invention, whereby the subject mounts an immune response against a pericyte of a vasculature of the solid tumor, thereby impeding a growth of a MAGE-b expressing breast cancer tumor in a subject.

In another embodiment, the present invention provides a method of overcoming an immune tolerance of a subject to a MAGE-b expressing breast cancer tumor, comprising administering to the subject an immunogenic composition comprising a nucleotide molecule of the present invention, whereby the subject mounts an immune response against a pericyte of a vasculature of the solid tumor, thereby overcoming an immune tolerance of a subject to a MAGE-b expressing breast cancer tumor.

“Tolerance” refers, in another embodiment, to a lack of responsiveness of the host to an antigen. In another embodiment, the term refers to a lack of detectable responsiveness of the host to an antigen. In another embodiment, the term refers to a lack of immunogenicity of an antigen in a host. In another embodiment, tolerance is measured by lack of responsiveness in an in vitro CTL killing assay. In another embodiment, tolerance is measured by lack of responsiveness in a delayed-type hypersensitivity assay. In another embodiment, tolerance is measured by lack of responsiveness in any other suitable assay known in the art. In another embodiment, tolerance is determined or measured as depicted in the Examples herein. Each possibility represents another embodiment of the present invention.

“Overcome” refers, in another embodiment, to a reversible of tolerance by a vaccine. In another embodiment, the term refers to conferment of detectable immune response by a vaccine. In another embodiment, overcoming of immune tolerance is determined or measured as depicted in the Examples herein. Each possibility represents another embodiment of the present invention.

In another embodiment, fusion proteins of the present invention need not be expressed by LM, but rather can be expressed and isolated from other vectors and cell systems used for protein expression and isolation.

As provided herein, and LLO-E7 fusion exhibits significant therapeutic efficacy. In these experiments, a vaccinia vector that expresses E7 as a fusion protein with a non-hemolytic truncated form of LLO was constructed. Expression of the LLO-E7 fusion product by plaque purified vaccinia was verified by Western blot using an antibody directed against the LLO protein sequence. Vac-LLO-E7 was demonstrated to produce CD8⁺ T cells specific to LLO and E7 was determined using the LLO (91-99) and E7 (49-57) epitopes of Balb/c and C57/BL6 mice, respectively. Results were confirmed in a chromium release assay.

Thus, expression of an antigen, e.g. MAGE-b, as a fusion protein with a non-hemolytic truncated form of LLO, ActA, or a PEST-like sequence in host cell systems in Listeria and host cell systems other than Listeria results in enhanced immunogenicity of the antigen. While comparative experiments were performed with vaccinia, a multitude of other plasmids and expression systems which can be used to express these fusion proteins are known. For example, bacterial vectors useful in the present invention include, but are not limited to Salmonella sp., Shigella sp., BCG, L. monocytogenes and S. gordonii. In addition the fusion proteins can be delivered by recombinant bacterial vectors modified to escape phagolysosomal fusion and live in the cytoplasm of the cell. Viral vectors useful in the present invention include, but are not limited to, Vaccinia, Avipox, Adenovirus, AAV, Vaccinia virus NYVAC, Modified vaccinia strain Ankara (MVA), Semliki Forest virus, Venezuelan equine encephalitis virus, herpes viruses, and retroviruses. Naked DNA vectors can also be used.

“MAGE-b peptide” refers, in another embodiment, to a full-length MAGE-b protein. In another embodiment, the term refers to a fragment of a MAGE-b protein. Each possibility represents a separate embodiment of the present invention.

In another embodiment, the MAGE-b protein that is the source of a MAGE-b peptide of methods and compositions of the present invention is a MAGE-b1 protein. In another embodiment, the MAGE-b protein is a MAGE-b2 protein. In another embodiment, the MAGE-b protein is a MAGE-b3 protein. In another embodiment, the MAGE-b protein is a MAGE-b4 protein.

In another embodiment, the MAGE-b protein that is the source of a MAGE-b peptide of methods and compositions of the present invention is a human MAGE-b protein. In another embodiment, the MAGE-b protein is a mouse MAGE-b protein. In another embodiment, the MAGE-b protein is a rodent MAGE-b protein. In another embodiment, 1 of the above MAGE-b protein is also referred to in the art as a “Mage-b protein.” In another embodiment, the MAGE-b protein is a MAGE-b protein of any other species known in the art. Each possibility represents a separate embodiment of the present invention.

In another embodiment, the MAGE-b protein has the sequence:

MPRGQKSKLRAREKRRKAREETQGLKVAHATAAEKEECPSSSPVLGDTPTSSPAA GIPQKPQGAPPTTTAAAAVSCTESDEGAKCQGEENASFSQATTSTESSVKDPVAWEAGML MHFILRKYKMREPIMKADMLKVVDEKYKDHFTEILNGASRRLELVFGLDLKEDNPSGHT YTLVSKLNLTNDGNLSNDWDFPRNGLLMPLLGVIFLKGNSATEEEIWKFMNVLGAYDGE EHLIYGEPRKFITQDLVQEKYLKYEQVPNSDPPRYQFLWGPRAYAETTKMKVLEFLAKM NGATPRDFPSHYEEALRDEEERAQVRSSVRARRRTTATTFRARSRAPFSRSSHPM (SEQ ID No: 25). In another embodiment, the MAGE-b protein is a homologue of SEQ ID No: 25. In another embodiment, the MAGE-b protein is a variant of SEQ ID No: 25. In another embodiment, the MAGE-b protein is an isomer of SEQ ID No: 25. In another embodiment, the MAGE-b protein is a fragment of SEQ ID No: 25. Each possibility represents a separate embodiment of the present invention.

In another embodiment, the MAGE-b protein is encoded by a nucleotide molecule having the sequence:

atgcctcggggtcagaagagtaagctccgtgctcgtgagaaacgccgcaaggcgcgagaggagacccagggtctcaaggttgc tcacgccactgcagcagagaaagaggagtgcccctcctcctctcctgttttaggggatactcccacaagctcccctgctgctggcattccccag aagcctcagggagctccacccaccaccactgctgctgcagctgtgtcatgtaccgaatctgacgaaggtgccaaatgccaaggtgaggaaa atgcaagtttctcccaggccacaacatccactgagagctcagtcaaagatcctgtagcctgggaggcaggaatgctgatgcacttcattctacg taagtataaaatgagagagcccattatgaaggcagatatgctgaaggttgttgatgaaaagtacaaggatcacttcactgagatcctcaatgga gcctctcgccgcttggagctcgtctttggccttgatttgaaggaagacaaccctagtggccacacctacaccctcgtcagtaagctaaacctca ccaatgatggaaacctgagcaatgattgggactttcccaggaatgggcttctgatgcctctcctgggtgtgatcttcttaaagggcaactctgcc accgaggaagagatctggaaattcatgaatgtgttgggagcctatgatggagaggagcacttaatctatggggaaccccgtaagttcatcacc caagatctggtgcaggaaaaatatctgaagtacgagcaggtgcccaacagtgatcccccacgctatcaattcctatggggtccgagagcctat gctgaaaccaccaagatgaaagtcctcgagtttttggccaagatgaatggtgccactcccgtgacttcccatcccattatgaagaggctttgag agatgaggaagagagagcccaagtccgatccagtgttagagccaggcgtcgcactactgccacgacttttagagcgcgttctagagccccat tcagcaggtcctcccaccccatgtga (SEQ ID No: 26). In another embodiment, the MAGE-b protein is encoded by a homologue of SEQ ID No: 26. In another embodiment, the MAGE-b protein is encoded by a variant of SEQ ID No: 26. In another embodiment, the MAGE-b protein is encoded by an isomer of SEQ ID No: 26. In another embodiment, the MAGE-b protein is encoded by a fragment of SEQ ID No: 26. Each possibility represents a separate embodiment of the present invention.

In another embodiment, the MAGE-b protein has the sequence:

MPRGQKSKLRAREKRRKARDETRGLNVPQVTEAEEEEAPCCSSSVSGGAASSSPA AGIPQEPQRAPTTAAAAAAGVSSTKSKKGAKSHQGEKNASSSQASTSTKSPSEDPLTRKS GSLVQFLLYKYKIKKSVTKGEMLKIVGKRFREHFPEILKKASEGLSVVFGLELNKVNPNG HTYTFIDKVDLTDEESLLSSWDFPRRKLLMPLLGVIFLNGNSATEEEIWEFLNMLGVYDGE EHSVFGEPWKLITKDLVQEKYLEYKQVPSSDPPRFQFLWGPRAYAETSKMKVLEFLAKV NGTTPCAFPTHYEEALKDEEKAGV (SEQ ID No: 27) In another embodiment, the MAGE-b protein is a homologue of SEQ ID No: 27. In another embodiment, the MAGE-b protein is a variant of SEQ ID No: 27. In another embodiment, the MAGE-b protein is an isomer of SEQ ID No: 27. In another embodiment, the MAGE-b protein is a fragment of SEQ ID No: 27. Each possibility represents a separate embodiment of the present invention.

In another embodiment, the MAGE-b protein has the sequence:

MPRGQKSTLHAREKRQQTRGQTQDHQGAQITATNKKKVSFSSPLILGATIQKKSA GRSRSALKKPQRALSTTTSVDVSYKKSYKGANSKIEKKQSFSQGLSSTVQSRTDPLIMKTN MLVQFLMEMYKMKKPIMKADMLKIVQKSHKNCFPEILKKASFNMEVVFGVDLKKVDST KDSYVLVSKMDLPNNGTVTRGRGFPKTGLLLNLLGVIFMKGNCATEEKIWEFLNKMRIY DGKKHFIFGEPRKLITQDLVKLKYLEYRQVPNSNPARYEFLWGPRAHAETSKMKVLEFW AKVNKTVPSAFQFWYEEALRDEEERVQAAAMLNDGSSAMGRKCSKAKASSSSHA (SEQ ID No: 28). In another embodiment, the MAGE-b protein is a homologue of SEQ ID No: 28. In another embodiment, the MAGE-b protein is a variant of SEQ ID No: 28. In another embodiment, the MAGE-b protein is an isomer of SEQ ID No: 28. In another embodiment, the MAGE-b protein is a fragment of SEQ ID No: 28. Each possibility represents a separate embodiment of the present invention.

In another embodiment, the MAGE-b protein that is the source of the MAGE-b peptide has the sequence:

(SEQ ID No: 29)

MPRGQKSKLRAREKRQRTRGQTQDLKVGQPTAAEKEESPSSSSSVLRDTA
SSSLAFGIPQEPQREPPTTSAAAAMSCTGSDKGDESQDEENASSSQASTS
TERSLKDSLTRKTKMLVQFLLYKYKMKEPTTKAEMLKIISKKYKEHFPEI
FRKVSQRTELVFGLALKEVNPTTHSYILVSMLGPNDGNQSSAWTLPRNGL
LMPLLSVIFLNGNCAREEEIWEFLNMLGIYDGKRHLIFGEPRKLITQDLV
QEKYLEYQQVPNSDPPRYQFLWGPRAHAETSKMKVLEFLAKVNDTTPNNF
PLLYEEALRDEEERAGARPRVAARRGTTAMTSAYSRATSSSSSQPM.

In another embodiment, the MAGE-b protein is a homologue of SEQ ID No: 29. In another embodiment, the MAGE-b protein is a variant of SEQ ID No: 29. In another embodiment, the MAGE-b protein is an isomer of SEQ ID No: 29. In another embodiment, the MAGE-b protein is a fragment of SEQ ID No: 29. Each possibility represents a separate embodiment of the present invention.

In another embodiment, the MAGE-b protein has the sequence:

(SEQ ID No: 30)

MPRGQKSKLRAREKRQRTRGQTQDLKVGQPTAAEKEESPSSSSSVLRDTA
SSSLAFGIPQEPQREPPTTSAAAAMSCTGSDKGDESQDEENASSSQASTS
TERSLKDSLTRKTKMLVQFLLYKYKMKEPTTKAEMLKIISKKYKEHFPEI
FRKVSQRTELVFGLALKEVNPTTHSYILVSMLGPNDGNQSSAWTLPRNGL
LMPLLSVIFLNGNCAREEEIWEFLNMLGIYDGKRHLIFGEPRKLITQDLV
QEKYLEYQQVPNSDPPRYQFLWGPRAHAETSKMKVLEFLAKVNDTTPNNF
PLLYEEALRDEEERAGARPRVAARRGTTAMTSAYSRATSSSSSQPM.

In another embodiment, the MAGE-b protein is a homologue of SEQ ID No: 30. In another embodiment, the MAGE-b protein is a variant of SEQ ID No: 30. In another embodiment, the MAGE-b protein is an isomer of SEQ ID No: 30. In another embodiment, the MAGE-b protein is a fragment of SEQ ID No: 30. Each possibility represents a separate embodiment of the present invention.

In another embodiment, the MAGE-b protein is encoded by a nucleotide molecule having the sequence:

aggatttcatttgctcttctccaggaaccacatcacctgcccttctgcctacactcctgcctgctgtgcctaaccacagccatcatgcct cggggtcagaagagtaagctccgtgccgtgagaaacgccagcggacccgtggtcagacccaggatctcaaggttggtcagcctactgca gcagagaaagaagagtctccttcctcttcctcatctgttttgagggatactgcctccagctcccttgcttttggcattccccaggagcctcagaga gagccacccaccacctctgctgctgcagctatgtcatgcactggatctgataaaggcgacgagagccaagatgaggaaaatgcaagttcctc ccaggcctcaacatccactgagagatcactcaaagattctctaaccaggaagacgaagatgttagtgcagttcctgctgtacaagtataaaatg aaagagcccactacaaaggcagaaatgctgaagatcatcagcaaaaagtacaaggagcacttccctgagatcttcaggaaagtctctcagcg cacggagctggtctttggccttgccttgaaggaggtcaaccccaccactcactcctacatcctcgtcagcatgctaggccccaacgatggaaa ccagagcagtgcctggacccttccaaggaatgggcttctgatgcctctactgagtgtgatcttcttaaatggcaactgtgcccgtgaagaggaa atctgggaattcctgaatatgctggggatctatgatggaaagaggcaccttatctttggggaaccccgaaagctcatcacccaagatctggtgc aggaaaaatatctggaataccagcaggtgcccaacagtgatcccccacgctatcaattcctgtggggtccaagagctcatgcagaaaccagc aagatgaaagtcctggagtttttggccaaggtgaatgacaccacccccaataacttcccactcctttatgaagaggctttgagagatgaagaag agagagctggagcccggcccagagttgcagccaggcgtggcactacagccatgactagtgcgtattccagggccacatccagtagctcttc ccaacccatgtgagatctaaggcaaattgttcactttgtggttgaaagacctgctgctttctctgttcctgtgatgcatgaataactcattgatttatct ctttgttgtattttccatgatgtttcttaaaatagaaagtttatttagattcagaatataaatttagaaatggcatgcatcacacatttattgctgtttatca ggttggtttagtgataataattttgtttttgaaatacaaatagaaaatcctgaaataatttttgtgatacagagcaaaataacacggcatgggagtaa ggttatccttagaaatttaaaataactccacagtaaaataggtagaatctgaagatagaaagggaagaaaagtaaaagttgctttattcgtggtttg tcttactcagttcagtctttttttgctcataaatttaaaagttacatacctggtttgcttagattattcaagaatgtggaggcctgggccaaggtcaatg acagtgtctccattgtcttccctccattaagagaagactttaagagatgagggagagagagccagagacagtgttgcaactgggcctggcatgt ttcagtgtggtgtccagcagtgtctcccactccttgtgaagtctgaggtatattctttacttttgattaagaaaacacttaaccttctaattaatggaga gccaaaggggagttggtgggaacaccatgtataacatatttgtatgtaaaatgatttatcttttctttttcctgtttttcagtgttctttttttaaattgtag atttatttagtttcagaatctaagtttatgaatggcatgaatcactcatttattaaaatatatcaggttggagagtgagaatttttgcattatgtaaaaca atttaaaaatcttttaagtctttttctgtgatctagaacaagataatatggcattggaatatggaatttgtgaaaaggaaattaccttgcaataaagttg gtgggaccaggaagtagagaaaaaaaaagtaaaa (SEQ ID No: 31). In another embodiment, the MAGE-b protein is encoded by a homologue of SEQ ID No: 31. In another embodiment, the MAGE-b protein is encoded by a variant of SEQ ID No: 31. In another embodiment, the MAGE-b protein is encoded by an isomer of SEQ ID No: 31. In another embodiment, the MAGE-b protein is encoded by a fragment of SEQ ID No: 31. Each possibility represents a separate embodiment of the present invention.

In another embodiment, the MAGE-b protein has the sequence:

MPRGQKSKTRSRAKRQQSRREVPVVQPTAEEAGSSPVDQSAGSSFPGGSAPQGVK TPGSFGAGVSCTGSGIGGRNAAVLPDTKSSDGTQAGTSIQHTLKDPIMRKASVLIEFLLDK FKMKEAVTRSEMLAVVNKKYKEQFPEIPRRTSARLELVFGLELKEIDPSTHSYLLVGKLG LSTEGSLSSNWGLPRTGLLMSVLGVIFMKGNRATEQEVWQFLHGVGVYAGKKHLIFGEP EEFIRDVVRENYLEYRQVPGSDPPSYEFLWGPRAHAETTKMKVLEVLAKVNGTVPSAFPN LYQLALRDQAGGVPRRRVQGKGVHSKAPSQKSSNM (SEQ ID No: 32). In another embodiment, the MAGE-b protein is a homologue of SEQ ID No: 32. In another embodiment, the MAGE-b protein is a variant of SEQ ID No: 32. In another embodiment, the MAGE-b protein is an isomer of SEQ ID No: 32. In another embodiment, the MAGE-b protein is a fragment of SEQ ID No: 32. Each possibility represents a separate embodiment of the present invention.

In another embodiment, the MAGE-b protein is encoded by a nucleotide molecule having the sequence:

atgcctaggggtcaaaagagtaagacccgctcccgtgcaaaacgacagcagtcacgcagggaggttccagtagttcagcccact gcagaggaagcagggtcttctcctgttgaccagagtgctgggtccagcttccctggtggttctgctcctcagggtgtgaaaacccctggatcttt tggtgcaggtgtatcctgcacaggctctggtataggtggtagaaatgctgctgtcctgcctgatacaaaaagttcagatggcacccaggcagg gacttccattcagcacacactgaaagatcctatcatgaggaaggctagtgtgctgatagaattcctgctagataagtttaagatgaaagaagcag ttacaaggagtgaaatgctggcagtagttaacaagaagtataaggagcaattccctgagatccccaggagaacttctgcacgcctagaattagt ctttggtcttgagttgaaggaaattgatcccagcactcattcctatttgctggtaggcaaactgggtctttccactgagggaagtttgagtagtaact gggggttgcctaggacaggtctcctaatgtctgtcctaggtgtgatcttcatgaagggtaaccgtgccactgagcaagaggtctggcaatttctg catggagtgggggtatatgctgggaagaagcacttgatctttggcgagcctgaggagtttataagagatgtagtgcgggaaaattacctggag taccgccaggtacctggcagtgatcccccaagctatgagttcctgtggggacccagagcccatgctgaaacaactaagatgaaagtcctgga agttttagctaaagtcaatggcacagtccctagtgccttccctaatctctaccagttggctcttagagatcaggcaggaggggtgccaagaagg agagttcaaggcaagggtgttcattccaaggccccatcccaaaagtcctctaacatgtaa (SEQ ID No: 33). In another embodiment, the MAGE-b protein is encoded by a homologue of SEQ ID No: 33. In another embodiment, the MAGE-b protein is encoded by a variant of SEQ ID No: 33. In another embodiment, the MAGE-b protein is encoded by an isomer of SEQ ID No: 33. In another embodiment, the MAGE-b protein is encoded by a fragment of SEQ ID No: 33. Each possibility represents a separate embodiment of the present invention.

In another embodiment, the MAGE-b protein is a transcript variant of a MAGE-b protein. Examples of transcript variants of a MAGE-b proteins are:

MAGE-b1, transcript variant 1, having the sequence:

MPRGQKSKLRAREKRRKAREETQGLKVAHATAAEKEECPSSSPVLGDTPTSSPAA GIPQKPQGAPPTTTAAAAVSCTESDEGAKCQGEENASFSQATTSTESSVKDPVAWEAGML MHFILRKYKMREPIMKADMLKVVDEKYKDHFTEILNGASRRLELVFGLDLKEDNPSGHT YTLVSKLNLTNDGNLSNDWDFPRNGLLMPLLGVIFLKGNSATEEEIWKFMNVLGAYDGE EHLIYGEPRKFITQDLVQEKYLKYEQVPNSDPPRYQFLWGPRAYAETTKMKVLEFLAKM NGATPRDFPSHYEEALRDEEERAQVRSSVRARRRTTATTFRARSRAPFSRSSHPM (SEQ ID No: 41; GenBank Accession No: NM_{—}002363). In another embodiment, the MAGE-b protein is a homologue of SEQ ID No: 41. In another embodiment, the MAGE-b protein is a variant of SEQ ID No: 41. In another embodiment, the MAGE-b protein is an isomer of SEQ ID No: 41. In another embodiment, the MAGE-b protein is a fragment of SEQ ID No: 41. Each possibility represents a separate embodiment of the present invention.

MAGE-b1, transcript variant 2, having the sequence:

MPRGQKSKLRAREKRRKAREETQGLKVAHATAAEKEECPSSSPVLGDTPTSSPAA GIPQKPQGAPPTTTAAAAVSCTESDEGAKCQGEENASFSQATTSTESSVKDPVAWEAGML MHFILRKYKMREPIMKADMLKVVDEKYKDHFTEILNGASRRLELVFGLDLKEDNPSGHT YTLVSKLNLTNDGNLSNDWDFPRNGLLMPLLGVIFLKGNSATEEEIWKFMNVLGAYDGE EHLIYGEPRKFITQDLVQEKYLKYEQVPNSDPPRYQFLWGPRAYAETTKMKVLEFLAKM NGATPRDFPSHYEEALRDEEERAQVRSSVRARRRTTATTFRARSRAPFSRSSHPM (SEQ ID No: 42; GenBank Accession No: NM_{—}177404). In another embodiment, the MAGE-b protein is a homologue of SEQ ID No: 42. In another embodiment, the MAGE-b protein is a variant of SEQ ID No: 42. In another embodiment, the MAGE-b protein is an isomer of SEQ ID No: 42. In another embodiment, the MAGE-b protein is a fragment of SEQ ID No: 42. Each possibility represents a separate embodiment of the present invention.

MAGE-b1, transcript variant 3, having the sequence:

MPRGQKSKLRAREKRRKAREETQGLKVAHATAAEKEECPSSSPVLGDTPTSSPAA GIPQKPQGAPPTTTAAAAVSCTESDEGAKCQGEENASFSQATTSTESSVKDPVAWEAGML MHFILRKYKMREPIMKADMLKVVDEKYKDHFTEILNGASRRLELVFGLDLKEDNPSGHT YTLVSKLNLTNDGNLSNDWDFPRNGLLMPLLGVIFLKGNSATEEEIWKFMNVLGAYDGE EHLIYGEPRKFITQDLVQEKYLKYEQVPNSDPPRYQFLWGPRAYAETTKMKVLEFLAKM NGATPRDFPSHYEEALRDEEERAQVRSSVRARRRTTATTFRARSRAPFSRSSHPM (SEQ ID No: 43; GenBank Accession No: NM_{—}177415). In another embodiment, the MAGE-b protein is a homologue of SEQ ID No: 43. In another embodiment, the MAGE-b protein is a variant of SEQ ID No: 43. In another embodiment, the MAGE-b protein is an isomer of SEQ ID No: 43. In another embodiment, the MAGE-b protein is a fragment of SEQ ID No: 43. Each possibility represents a separate embodiment of the present invention.

In another embodiment, the MAGE-b protein has a sequence selected from the AA and nucleotide sequences set forth in GenBank Accession No BD190644-661.

In another embodiment, the MAGE-b protein of methods and compositions of the present invention is required for a tumor phenotype. In another embodiment, the MAGE-b protein is necessary for transformation of a tumor cell. In another embodiment, tumor cells that lose expression of the MAGE-b protein lose their uncontrolled growth, invasiveness, or another feature of malignancy. Each possibility represents a separate embodiment of the present invention.

In another embodiment, a MAGE-b protein of methods and compositions of the present invention shares complete homology with the MAGE-b peptide (throughout the length of the peptide) expressed by the Listerial vector. In another embodiment, the MAGE-b protein is highly homologous (throughout the length of the peptide) to the MAGE-b peptide expressed by the Listerial vector. “Highly homologous” refers, in another embodiment, to a homology of greater than 90%. In another embodiment, the term refers to a homology of greater than 92%. In another embodiment, the term refers to a homology of greater than 93%. In another embodiment, the term refers to a homology of greater than 94%. In another embodiment, the term refers to a homology of greater than 95%. In another embodiment, the term refers to a homology of greater than 96%. In another embodiment, the term refers to a homology of greater than 97%. In another embodiment, the term refers to a homology of greater than 98%. In another embodiment, the term refers to a homology of greater than 99%. In another embodiment, the term refers to a homology of 100%. Each possibility represents a separate embodiment of the present invention.

Each MAGE-b protein represents a separate embodiment of the present invention.

The MAGE-b peptide of methods and compositions of the present invention comprises, in another embodiment, the sequence:

KASVLIEFLLDKFKMKEAVTRSEMLAVVNKKYKEQFPEIPRRTSARLELVFGLELK EIDPSTHSYLLVGKLGLSTEGSLSSNWGLPRTGLLMSVLGVIFMKGNRATEQEVWQFLHG (SEQ ID No: 34), which is AA 105-220 from SEQ ID No: 32. In another embodiment, the MAGE-b peptide comprises a sequence of a homologous MAGE-b protein, corresponding to SEQ ID No: 34. In another embodiment, the homologous MAGE-b protein is a MAGE-b transcript variant. In another embodiment, the MAGE-b peptide comprises a sequence homologous to SEQ ID No: 34. In another embodiment, the MAGE-b peptide comprises a sequence that is a variant of SEQ ID No: 34. In another embodiment, the MAGE-b protein comprises a sequence that is a fragment of SEQ ID No: 34. Each possibility represents a separate embodiment of the present invention.

Methods of identifying corresponding sequences in related proteins are well known in the art, and include, for example, AA sequence alignment. In another embodiment, the MAGE-b peptide comprises the sequence:

EAGMLMHFILRKYKMREPIMKADMLKVVDEKYKDHFTEILNGASRRLELVFGLD LKEDNPSGHTYTLVSKLNLTNDGNLSNDWDFPRNGLLMPLLGVIFLKGNSATEEEIWKF MNV (SEQ ID No: 35), which is a homo sapiens MAGEB1 sequence corresponding to SEQ ID No: 34. In another embodiment, the MAGE-b peptide comprises a sequence of a homologous MAGE-b protein, corresponding to SEQ ID No: 35. In another embodiment, the homologous MAGE-b protein is a MAGE-b transcript variant. In another embodiment, the MAGE-b peptide comprises a sequence homologous to SEQ ID No: 35. In another embodiment, the MAGE-b peptide comprises a sequence that is a variant of SEQ ID No: 35. In another embodiment, the MAGE-b protein comprises a sequence that is a fragment of SEQ ID No: 35. Each possibility represents a separate embodiment of the present invention.

In another embodiment, the MAGE-b peptide comprises the sequence:

KTKMLVQFLLYKYKMKEPTTKAEMLKIISKKYKEHFPEIFRKVSQRTELVFGLALK EVNPTTHSYILVSMLGPNDGNQSSAWTLPRNGLLMPLLSVIFLNGNCAREEEIWEFLNM (SEQ ID No: 36), which is a homo sapiens MAGEB4 sequence] corresponding to SEQ ID No: 34. In another embodiment, the MAGE-b peptide comprises a sequence of a homologous MAGE-b protein, corresponding to SEQ ID No: 36. In another embodiment, the homologous MAGE-b protein is a MAGE-b transcript variant. In another embodiment, the MAGE-b peptide comprises a sequence homologous to SEQ ID No: 36. In another embodiment, the MAGE-b peptide comprises a sequence that is a variant of SEQ ID No: 36. In another embodiment, the MAGE-b protein comprises a sequence that is a fragment of SEQ ID No: 36. Each possibility represents a separate embodiment of the present invention.

In another embodiment, the MAGE-b peptide comprises the sequence:

KSGSLVQFLLYKYKIKKSVTKGEMLKIVGKRFREHFPEILKKASEGLSVVFGLELN KVNPNGHTYTFIDKVDLTDEESLLSSWDFPRRKLLMPLLGVIFLNGNSATEEEIWEFLNM (SEQ ID No: 37), which is a homo sapiens MAGEB2 sequence corresponding to SEQ ID No: 34. In another embodiment, the MAGE-b peptide comprises a sequence of a homologous MAGE-b protein, corresponding to SEQ ID No: 37. In another embodiment, the homologous MAGE-b protein is a MAGE-b transcript variant. In another embodiment, the MAGE-b peptide comprises a sequence homologous to SEQ ID No: 37. In another embodiment, the MAGE-b peptide comprises a sequence that is a variant of SEQ ID No: 37. In another embodiment, the MAGE-b protein comprises a sequence that is a fragment of SEQ ID No: 37. Each possibility represents a separate embodiment of the present invention.

In another embodiment, the MAGE-b peptide comprises the sequence:

KTNMLVQFLMEMYKMKKPIMKADMLKIVQKSHKNCFPEILKKASFNMEVVFGV DLKKVDSTKDSYVLVSKMDLPNNGTVTRGRGFPKTGLLLNLLGVIFMKGNCATEEKIWE FLNK (SEQ ID No: 38), which is a homo sapiens MAGEB3 sequence corresponding to SEQ ID No: 34. In another embodiment, the MAGE-b peptide comprises a sequence of a homologous MAGE-b protein, corresponding to SEQ ID No: 38. In another embodiment, the homologous MAGE-b protein is a MAGE-b transcript variant. In another embodiment, the MAGE-b peptide comprises a sequence homologous to SEQ ID No: 38. In another embodiment, the MAGE-b peptide comprises a sequence that is a variant of SEQ ID No: 38. In another embodiment, the MAGE-b protein comprises a sequence that is a fragment of SEQ ID No: 38. Each possibility represents a separate embodiment of the present invention.

In another embodiment, the MAGE-b peptide comprises the sequence:

EAGMLMHFILRKYKMREPIMKADMLKVVDEKYKDHFTEILNGASRRLELVFGLD LKEDNPSGHTYTLVSKLNLTNDGNLSNDWDFPRNGLLMPLLGVIFLKGNSATEEEIWKF MNV (SEQ ID No: 50), which is the sequence of MAGE-b1, transcript variants 1-3 (SEQ ID No: 41-43, as disclosed herein) that corresponds to SEQ ID No: 34. In another embodiment, the MAGE-b peptide comprises a sequence of a homologous MAGE-b protein, corresponding to SEQ ID No: 50. In another embodiment, the homologous MAGE-b protein is a MAGE-b transcript variant. In another embodiment, the MAGE-b peptide comprises a sequence homologous to SEQ ID No: 50. In another embodiment, the MAGE-b peptide comprises a sequence that is a variant of SEQ ID No: 50. In another embodiment, the MAGE-b protein comprises a sequence that is a fragment of SEQ ID No: 50. Each possibility represents a separate embodiment of the present invention.

In another embodiment, the MAGE-b peptide comprises the sequence:

MKGNRATEQEVWQFLHGVGVYAGKKHLIFGEPEEFIRDVVRENYLEYRQVPGSD PPSYEFLWGPRAHAETTKMKVLEVLAKVNGTVPSAFPNLYQLALRDQAGGVPRRRVQG KGVHSKAPSQKSSNM (SEQ ID No: 39), which is AA 204-330 from SEQ ID No: 32. In another embodiment, the MAGE-b peptide comprises the sequence: In another embodiment, the MAGE-b peptide comprises a sequence of a homologous MAGE-b protein, corresponding to SEQ ID No: 39. In another embodiment, the homologous MAGE-b protein is a MAGE-b transcript variant. In another embodiment, the MAGE-b peptide comprises a sequence homologous to SEQ ID No: 39. In another embodiment, the MAGE-b peptide comprises a sequence that is a variant of SEQ ID No: 39. In another embodiment, the MAGE-b protein comprises a sequence that is a fragment of SEQ ID No: 39. Each possibility represents a separate embodiment of the present invention.

In another embodiment, the MAGE-b peptide comprises the sequence:

MPRGQKSKTRSRAKRQQSRREVPVVQPTAEEAGSSPVDQSAGSSFPGGSAPQGVK TPGSFGAGVSCTGSGIGGRNAAVLPDTKSSDGTQAGTSIQHTLKDPIMRKASVLIEFLLDK F (SEQ ID No: 40), which is AA 2-117 from SEQ ID No: 32. In another embodiment, the MAGE-b peptide comprises a sequence of a homologous MAGE-b protein, corresponding to SEQ ID No: 40. In another embodiment, the homologous MAGE-b protein is a MAGE-b transcript variant. In another embodiment, the MAGE-b peptide comprises a sequence homologous to SEQ ID No: 40. In another embodiment, the MAGE-b peptide comprises a sequence that is a variant of SEQ ID No: 40. In another embodiment, the MAGE-b protein comprises a sequence that is a fragment of SEQ ID No: 40. Each possibility represents a separate embodiment of the present invention.

The MAGE-b peptide of methods and compositions of the present invention is, in another embodiment, 200-400 amino acids (AA) in length. In another embodiment, the MAGE-b peptide is about 117-127 AA long. In another embodiment, the length is 100-330 AA. In another embodiment, the length is 110-330 AA. In another embodiment, the length is 120-330 AA. In another embodiment, the length is 130-330 AA. In another embodiment, the length is 140-330 AA. In another embodiment, the length is 150-330 AA. In another embodiment, the length is 160-330 AA. In another embodiment, the length is 175-330 AA. In another embodiment, the length is 190-330 AA. In another embodiment, the length is 200-330 AA. In another embodiment, the length is 210-330 AA. In another embodiment, the length is 220-330 AA. In another embodiment, the length is 230-330 AA. In another embodiment, the length is 240-330 AA. In another embodiment, the length is 250-330 AA. In another embodiment, the length is 260-330 AA. In another embodiment, the length is 270-330 AA. In another embodiment, the length is 300-330 AA.

In another embodiment, the length is about 175 AA. In another embodiment, the length is about 200 AA. In another embodiment, the length is about 220 AA. In another embodiment, the length is about 240 AA. In another embodiment, the length is about 260 AA. In another embodiment, the length is about 280 AA. In another embodiment, the length is about 300 AA. In another embodiment, the length is about 320 AA.

Each length represents a separate embodiment of the present invention.

In another embodiment, the MAGE-b peptide of methods and compositions of the present invention consists of AA 2-117 of SEQ ID No: 32 or a corresponding fragment thereof of a homologous protein. In another embodiment, the MAGE-b peptide consists of AA 105-220 of SEQ ID No: 32 or a corresponding fragment thereof of a homologous protein. In another embodiment, the MAGE-b peptide consists of AA 204-330 of SEQ ID No: 32 or a corresponding fragment thereof of a homologous protein. In another embodiment, the MAGE-b peptide consists of another fragment of a MAGE-b protein. Each possibility represents a separate embodiment of the present invention.

In another embodiment, the MAGE-b peptide consists of about one-third to one-half of the MAGE-b protein. In another embodiment, the fragment consists of about one-tenth to one-fifth thereof. In another embodiment, the fragment consists of about one-fifth to one-fourth thereof. In another embodiment, the fragment consists of about one-fourth to one-third thereof. In another embodiment, the fragment consists of about one-third to one-half thereof. In another embodiment, the fragment consists of about one-half to three quarters thereof. In another embodiment, the fragment consists of about three quarters to the MAGE-b protein. In another embodiment, the fragment consists of about 5-10% thereof. In another embodiment, the fragment consists of about 10-15% thereof. In another embodiment, the fragment consists of about 15-20% thereof. In another embodiment, the fragment consists of about 20-25% thereof. In another embodiment, the fragment consists of about 25-30% thereof. In another embodiment, the fragment consists of about 30-35% thereof. In another embodiment, the fragment consists of about 35-40% thereof. In another embodiment, the fragment consists of about 45-50% thereof. In another embodiment, the fragment consists of about 50-55% thereof. In another embodiment, the fragment consists of about 55-60% thereof. In another embodiment, the fragment consists of about 5-15% thereof. In another embodiment, the fragment consists of about 10-20% thereof. In another embodiment, the fragment consists of about 15-25% thereof. In another embodiment, the fragment consists of about 20-30% thereof. In another embodiment, the fragment consists of about 25-35% thereof. In another embodiment, the fragment consists of about 30-40% thereof. In another embodiment, the fragment consists of about 35-45% thereof. In another embodiment, the fragment consists of about 45-55% thereof. In another embodiment, the fragment consists of about 50-60% thereof. In another embodiment, the fragment consists of about 55-65% thereof. In another embodiment, the fragment consists of about 60-70% thereof. In another embodiment, the fragment consists of about 65-75% thereof. In another embodiment, the fragment consists of about 70-80% thereof. In another embodiment, the fragment consists of about 5-20% thereof. In another embodiment, the fragment consists of about 10-25% thereof. In another embodiment, the fragment consists of about 15-30% thereof. In another embodiment, the fragment consists of about 20-35% thereof. In another embodiment, the fragment consists of about 25-40% thereof. In another embodiment, the fragment consists of about 30-45% thereof. In another embodiment, the fragment consists of about 35-50% thereof. In another embodiment, the fragment consists of about 45-60% thereof. In another embodiment, the fragment consists of about 50-65% thereof. In another embodiment, the fragment consists of about 55-70% thereof. In another embodiment, the fragment consists of about 60-75% thereof. In another embodiment, the fragment consists of about 65-80% thereof. In another embodiment, the fragment consists of about 70-85% thereof. In another embodiment, the fragment consists of about 75-90% thereof. In another embodiment, the fragment consists of about 80-95% thereof. In another embodiment, the fragment consists of about 85-100% thereof. In another embodiment, the fragment consists of about 5-25% thereof. In another embodiment, the fragment consists of about 10-30% thereof. In another embodiment, the fragment consists of about 15-35% thereof. In another embodiment, the fragment consists of about 20-40% thereof. In another embodiment, the fragment consists of about 30-50% thereof. In another embodiment, the fragment consists of about 40-60% thereof. In another embodiment, the fragment consists of about 50-70% thereof. In another embodiment, the fragment consists of about 60-80% thereof. In another embodiment, the fragment consists of about 70-90% thereof. In another embodiment, the fragment consists of about 80-100% thereof. In another embodiment, the fragment consists of about 5-35% thereof. In another embodiment, the fragment consists of about 10-40% thereof. In another embodiment, the fragment consists of about 15-45% thereof. In another embodiment, the fragment consists of about 20-50% thereof. In another embodiment, the fragment consists of about 30-60% thereof. In another embodiment, the fragment consists of about 40-70% thereof. In another embodiment, the fragment consists of about 50-80% thereof. In another embodiment, the fragment consists of about 60-90% thereof. In another embodiment, the fragment consists of about 70-100% thereof. In another embodiment, the fragment consists of about 5-45% thereof. In another embodiment, the fragment consists of about 10-50% thereof. In another embodiment, the fragment consists of about 20-60% thereof. In another embodiment, the fragment consists of about 30-70% thereof. In another embodiment, the fragment consists of about 40-80% thereof. In another embodiment, the fragment consists of about 50-90% thereof. In another embodiment, the fragment consists of about 60-100% thereof. In another embodiment, the fragment consists of about 5-55% thereof. In another embodiment, the fragment consists of about 10-60% thereof. In another embodiment, the fragment consists of about 20-70% thereof. In another embodiment, the fragment consists of about 30-80% thereof. In another embodiment, the fragment consists of about 40-90% thereof. In another embodiment, the fragment consists of about 50-100% thereof. In another embodiment, the fragment consists of about 5-65% thereof. In another embodiment, the fragment consists of about 10-70% thereof. In another embodiment, the fragment consists of about 20-80% thereof. In another embodiment, the fragment consists of about 30-90% thereof. In another embodiment, the fragment consists of about 40-100% thereof. In another embodiment, the fragment consists of about 5-75% thereof. In another embodiment, the fragment consists of about 10-80% thereof. In another embodiment, the fragment consists of about 20-90% thereof. In another embodiment, the fragment consists of about 30-100% thereof. In another embodiment, the fragment consists of about 10-90% thereof. In another embodiment, the fragment consists of about 20-100% thereof. In another embodiment, the fragment consists of about 10-100% thereof.

In another embodiment, the fragment consists of about 5% of the MAGE-b protein. In another embodiment, the fragment consists of about 6% thereof. In another embodiment, the fragment consists of about 8% thereof. In another embodiment, the fragment consists of about 10% thereof. In another embodiment, the fragment consists of about 12% thereof. In another embodiment, the fragment consists of about 15% thereof. In another embodiment, the fragment consists of about 18% thereof. In another embodiment, the fragment consists of about 20% thereof. In another embodiment, the fragment consists of about 25% thereof. In another embodiment, the fragment consists of about 30% thereof. In another embodiment, the fragment consists of about 35% thereof. In another embodiment, the fragment consists of about 40% thereof. In another embodiment, the fragment consists of about 45% thereof. In another embodiment, the fragment consists of about 50% thereof. In another embodiment, the fragment consists of about 55% thereof. In another embodiment, the fragment consists of about 60% thereof. In another embodiment, the fragment consists of about 65% thereof. In another embodiment, the fragment consists of about 70% thereof. In another embodiment, the fragment consists of about 75% thereof. In another embodiment, the fragment consists of about 80% thereof. In another embodiment, the fragment consists of about 85% thereof. In another embodiment, the fragment consists of about 90% thereof. In another embodiment, the fragment consists of about 95% thereof. In another embodiment, the fragment consists of about 100% thereof. Each possibility represents a separate embodiment of the present invention.

In another embodiment, the immunogenic fragment of SEQ ID No: 34-39 contained in a MAGE-b peptide of methods and compositions of the present invention is about 10-117 AA long. In another embodiment, the length is 15-117 AA. In another embodiment, the length is 20-117 AA. In another embodiment, the length is 30-117 AA. In another embodiment, the length is 40-117 AA. In another embodiment, the length is 50-117 AA. In another embodiment, the length is 60-117 AA. In another embodiment, the length is 70-117 AA. In another embodiment, the length is 80-117 AA. In another embodiment, the length is 90-117 AA. In another embodiment, the length is 100-117 AA. In another embodiment, the length is 10-100 AA. In another embodiment, the length is 15-100 AA. In another embodiment, the length is 20-100 AA. In another embodiment, the length is 30-100 AA. In another embodiment, the length is 40-100 AA. In another embodiment, the length is 50-100 AA. In another embodiment, the length is 60-100 AA. In another embodiment, the length is 70-100 AA. In another embodiment, the length is 10-80 AA. In another embodiment, the length is 15-80 AA. In another embodiment, the length is 20-80 AA. In another embodiment, the length is 30-80 AA. In another embodiment, the length is 40-80 AA. In another embodiment, the length is 50-80 AA. In another embodiment, the length is 60-80 AA. In another embodiment, the length is 70-80 AA. In another embodiment, the length is 10-60 AA. In another embodiment, the length is 15-60 AA. In another embodiment, the length is 20-60 AA. In another embodiment, the length is 30-60 AA. In another embodiment, the length is 40-60 AA. In another embodiment, the length is 50-60 AA. In another embodiment, the length is 10-50 AA. In another embodiment, the length is 15-50 AA. In another embodiment, the length is 20-50 AA. In another embodiment, the length is 30-50 AA. In another embodiment, the length is 40-50 AA. In another embodiment, the length is 10-40 AA. In another embodiment, the length is 15-40 AA. In another embodiment, the length is 20-40 AA. In another embodiment, the length is 30-40 AA. In another embodiment, the length is 10-30 AA. In another embodiment, the length is 15-30 AA. In another embodiment, the length is 20-30 AA. In another embodiment, the length is 5-20 AA. In another embodiment, the length is 10-20 AA. In another embodiment, the length is 15-20 AA.

In another embodiment, the length of the immunogenic fragment is about 10 AA. In another embodiment, the length is about 15 AA. In another embodiment, the length is about 20 AA. In another embodiment, the length is about 30 AA. In another embodiment, the length is about 40 AA. In another embodiment, the length is about 50 AA. In another embodiment, the length is about 60 AA. In another embodiment, the length is about 70 AA. In another embodiment, the length is about 80 AA. In another embodiment, the length is about 90 AA. In another embodiment, the length is about 100 AA.

In another embodiment, the present invention provides a method of reducing a size of a MAGE-b-expressing tumor, comprising administering a vaccine, immunogenic composition, or vector comprising a recombinant Listeria strain of the present invention, thereby reducing a size of a MAGE-b-expressing tumor. In another embodiment, a cell of the tumor expresses MAGE-b. Each possibility represents a separate embodiment of the present invention.

In another embodiment, the present invention provides a method of suppressing a formation of a MAGE-b-expressing tumor, comprising administering an effective amount of a vaccine comprising either: (a) a recombinant Listeria strain comprising an N-terminal fragment of a protein fused to a MAGE-b peptide; or (b) a recombinant nucleotide encoding the recombinant polypeptide, whereby the subject mounts an immune response against the MAGE-b-expressing tumor, thereby suppressing a formation of a MAGE-b-expressing tumor.

In another embodiment, the present invention provides a method of reducing a size of a MAGE-b-expressing tumor, comprising administering a vaccine, immunogenic composition, or vector comprising a recombinant polypeptide of the present invention, thereby reducing a size of a MAGE-b-expressing tumor. In another embodiment, a cell of the tumor expresses MAGE-b. Each possibility represents a separate embodiment of the present invention.

In another embodiment, the present invention provides a method of suppressing a formation of a MAGE-b-expressing tumor, comprising administering an effective amount of a vaccine comprising either: (a) a recombinant polypeptide comprising an N-terminal fragment of a protein fused to a MAGE-b peptide; or (b) a recombinant nucleotide encoding the recombinant polypeptide, whereby the subject mounts an immune response against the MAGE-b-expressing tumor, thereby suppressing a formation of a MAGE-b-expressing tumor.

In another embodiment, the present invention provides a method of reducing a size of a MAGE-b-expressing tumor, comprising administering a vaccine, immunogenic composition, or vector comprising a recombinant nucleotide molecule of the present invention, thereby reducing a size of a MAGE-b-expressing tumor. In another embodiment, a cell of the tumor expresses MAGE-b. Each possibility represents a separate embodiment of the present invention.

In another embodiment, the present invention provides a method of suppressing a formation of a MAGE-b-expressing tumor, comprising administering an effective amount of a vaccine comprising either: (a) a recombinant nucleotide molecule comprising an N-terminal fragment of a protein fused to a MAGE-b peptide; or (b) a recombinant nucleotide encoding the recombinant polypeptide, whereby the subject mounts an immune response against the MAGE-b-expressing tumor, thereby suppressing a formation of a MAGE-b-expressing tumor.

The non-MAGE-b peptide of methods and compositions of the present invention is, in another embodiment, a listeriolysin (LLO) peptide. In another embodiment, the non-MAGE-b peptide is an ActA peptide. In another embodiment, the non-MAGE-b peptide is a PEST-like sequence peptide. In another embodiment, the non-MAGE-b peptide is any other peptide capable of enhancing the immunogenicity of a MAGE-b peptide. Each possibility represents a separate embodiment of the present invention.

The LLO protein utilized to construct vaccines of the present invention has, in another embodiment, the sequence:

MKKIMLVFITLILVSLPIAQQTEAKDASAFNKENSISSMAPPASPPASPKTPIEKKHADEIDK YIQGLDYNKNNVLVYHGDAVTNVPPRKGYKDGNEYIVVEKKKKSINQNNADIQVVNAIS SLTYPGALVKANSELVENQPDVLPVKRDSLTLSIDLPGMTNQDNKIVVKNATKSNVNNA VNTLVERWNEKYAQAYPNVSAKIDYDDEMAYSESQLIAKFGTAFKAVNNSLNVNFGAIS EGKMQEEVISFKQIYYNVNVNEPTRPSRFFGKAVTKEQLQALGVNAENPPAYISSVAYGR QVYLKLSTNSHSTKVKAAFDAAVSGKSVSGDVELTNIIKNSSFKAVIYGGSAKDEVQIIDG NLGDLRDILKKGATFNRETPGVPIAYTTNFLKDNELAVIKNNSEYIETTSKAYTDGKINIDH SGGYVAQFNISWDEVNYDPEGNEIVQHKNWSENNKSKLAHFTSSIYLPGNARNINVYAKE CTGLAWEWWRTVIDDRNLPLVKNRNISIWGTTLYPKYSNKVDNPIE (GenBank Accession No. P13128; SEQ ID NO: 17; nucleic acid sequence is set forth in GenBank Accession No. X15127). The first 25 amino acids of the proprotein corresponding to this sequence are the signal sequence and are cleaved from LLO when it is secreted by the bacterium. Thus, in this embodiment, the full length active LLO protein is 504 residues long. In another embodiment, the LLO protein is a homologue of SEQ ID No: 17. In another embodiment, the LLO protein is a variant of SEQ ID No: 17. In another embodiment, the LLO protein is an isomer of SEQ ID No: 17. In another embodiment, the LLO protein is a fragment of SEQ ID No: 17. Each possibility represents a separate embodiment of the present invention.

In another embodiment, “LLO peptide” and “LLO fragment” refer to an N-terminal fragment of an LLO protein. In another embodiment, the terms refer to a full-length but non-hemolytic LLO protein. In another embodiment, the terms refer to a non-hemolytic protein containing a point mutation in cysteine 484 of sequence ID No: 17 or a corresponding residue thereof in a homologous LLO protein. Each possibility represents a separate embodiment of the present invention.

In another embodiment, the N-terminal fragment of an LLO protein utilized in compositions and methods of the present invention has the sequence:

MKKIMLVFITLILVSLPIAQQTEAKDASAFNKENSISSVAPPASPPASPKTPIEKKHADEIDK YIQGLDYNKNNVLVYHGDAVTNVPPRKGYKDGNEYIVVEKKKKSINQNNADIQVVNAIS SLTYPGALVKANSELVENQPDVLPVKRDSLTLSIDLPGMTNQDNKIVVKNATKSNVNNA VNTLVERWNEKYAQAYSNVSAKIDYDDEMAYSESQLIAKFGTAFKAVNNSLNVNFGAIS EGKMQEEVISFKQIYYNVNVNEPTRPSRFFGKAVTKEQLQALGVNAENPPAYIS SVAYGR QVYLKLSTNSHSTKVKAAFDAAVSGKSVSGDVELTNIIKNSSFKAVIYGGSAKDEVQIIDG NLGDLRDILKKGATFNRETPGVPIAYTTNFLKDNELAVIKNNSEYIETTSKAYTDGKINIDH SGGYVAQFNISWDEVNYD (SEQ ID NO: 18). In another embodiment, the LLO fragment is a homologue of SEQ ID No: 18. In another embodiment, the LLO fragment is a variant of SEQ ID No: 18. In another embodiment, the LLO fragment is an isomer of SEQ ID No: 18. In another embodiment, the LLO fragment is a fragment of SEQ ID No: 18. Each possibility represents a separate embodiment of the present invention.

In another embodiment, the LLO fragment has the sequence: MKKIMLVFITLILVSLPIAQQTEAKDASAFNKENSISSVAPPASPPASPKTPIEKKHADEIDK YIQGLDYNKNNVLVYHGDAVTNVPPRKGYKDGNEYIVVEKKKKSINQNNADIQVVNAIS SLTYPGALVKANSELVENQPDVLPVKRDSLTLSIDLPGMTNQDNKIVVKNATKSNVNNA VNTLVERWNEKYAQAYSNVSAKIDYDDEMAYSESQLIAKFGTAFKAVNNSLNVNFGAIS EGKMQEEVISFKQIYYNVNVNEPTRPSRFFGKAVTKEQLQALGVNAENPPAYISSVAYGR QVYLKLSTNSHSTKVKAAFDAAVSGKSVSGDVELTNIIKNSSFKAVIYGGSAKDEVQIIDG NLGDLRDILKKGATFNRETPGVPIAYTTNFLKDNELAVIKNNSEYIETTSKAYTD (SEQ ID NO: 19). In another embodiment, the LLO fragment is a homologue of SEQ ID No: 19. In another embodiment, the LLO fragment is a variant of SEQ ID No: 19. In another embodiment, the LLO fragment is an isomer of SEQ ID No: 19. In another embodiment, the LLO fragment is a fragment of SEQ ID No: 19. Each possibility represents a separate embodiment of the present invention.

In another embodiment, the LLO fragment is any other LLO fragment known in the art. Each possibility represents a separate embodiment of the present invention.

“ActA peptide” refers, in another embodiment, to a full-length ActA protein. In another embodiment, the term refers to an ActA fragment. Each possibility represents a separate embodiment of the present invention.

The ActA fragment of methods and compositions of the present invention is, in another embodiment, an N-terminal ActA fragment. In another embodiment, the fragment is any other type of ActA fragment known in the art. Each possibility represents a separate embodiment of the present invention.

In another embodiment, the N-terminal fragment of an ActA protein has the sequence: MRAMMVVFITANCITINPDIIFAATDSEDSSLNTDEWEEEKTEEQPSEVNTGPRYETAREV SSRDIKELEKSNKVRNTNKADLIAMLKEKAEKGPNINNNNSEQTENAAINEEASGADRPAI QVERRHPGLPSDSAAEIKKRRKAIASSDSELESLTYPDKPTKVNKKKVAKESVADASESDL DS SMQSADES SPQPLKANQQPFFPKVFKKIKDAGKWVRDKIDENPEVKKAIVDKSAGLID QLLTKKKSEEVNASDFPPPPTDEELRLALPETPMLLGFNAPATSEPSSFEFPPPPTDEELRLA LPETPMLLGFNAPATSEPSSFEFPPPPTEDELEIIRETASSLDSSFTRGDLASLRNAINRHSQN FSDFPPIPTEEELNGRGGRP (SEQ ID No: 15). In another embodiment, the ActA fragment comprises SEQ ID No: 15. In another embodiment, the ActA fragment is a homologue of SEQ ID No: 15. In another embodiment, the ActA fragment is a variant of SEQ ID No: 15. In another embodiment, the ActA fragment is an isomer of SEQ ID No: 15. In another embodiment, the ActA fragment is a fragment of SEQ ID No: 15. Each possibility represents a separate embodiment of the present invention.

In another embodiment, the N-terminal fragment of an ActA protein has the sequence: MRAMMVVFITANCITINPDIIFAATDSEDSSLNTDEWEEEKTEEQPSEVNTGPRYETAREV SSRDIKELEKSNKVRNTNKADLIAMLKEKAEKGPNINNN (SEQ ID No: 44). In another embodiment, the ActA fragment is a homologue of SEQ ID No: 44. In another embodiment, the ActA fragment is a variant of SEQ ID No: 44. In another embodiment, the ActA fragment is an isomer of SEQ ID No: 44. Each possibility represents a separate embodiment of the present invention.

In another embodiment, the ActA fragment of methods and compositions of the present invention comprises a PEST-like sequence. In another embodiment, the PEST-like sequence contained in the ActA fragment is selected from SEQ ID No: 2-5. In another embodiment, the ActA fragment comprises at least 2 of the PEST-like sequences set forth in SEQ ID No: 2-5. In another embodiment, the ActA fragment comprises at least 3 of the PEST-like sequences set forth in SEQ ID No: 2-5. In another embodiment, the ActA fragment comprises the 4 PEST-like sequences set forth in SEQ ID No: 2-5. Each possibility represents a separate embodiment of the present invention.

In another embodiment, the N-terminal ActA fragment is encoded by a nucleotide molecule having the sequence SEQ ID NO: 16:

atgcgtgcgatgatggtggttttcattactgccaattgcattacgattaaccccgacataatatttgcagcgacagatagcgaagattct agtctaaacacagatgaatgggaagaagaaaaaacagaagagcaaccaagcgaggtaaatacgggaccaagatacgaaactgcacgtga agtaagttcacgtgatattaaagaactagaaaaatcgaataaagtgagaaatacgaacaaagcagacctaatagcaatgttgaaagaaaaagc agaaaaaggtccaaatatcaataataacaacagtgaacaaactgagaatgcggctataaatgaagaggcttcaggagccgaccgaccagct atacaagtggagcgtcgtcatccaggattgccatcggatagcgcagcggaaattaaaaaaagaaggaaagccatagcatcatcggatagtga gcttgaaagccttacttatccggataaaccaacaaaagtaaataagaaaaaagtggcgaaagagtcagttgcggatgcttctgaaagtgactta gattctagcatgcagtcagcagatgagtcttcaccacaacctttaaaagcaaaccaacaaccatttttccctaaagtatttaaaaaaataaaagat gcggggaaatgggtacgtgataaaatcgacgaaaatcctgaagtaaagaaagcgattgttgataaaagtgcagggttaattgaccaattattaa ccaaaaagaaaagtgaagaggtaaatgcttcggacttcccgccaccacctacggatgaagagttaagacttgctttgccagagacaccaatg cttcttggttttaatgctcctgctacatcagaaccgagctcattcgaatttccaccaccacctacggatgaagagttaagacttgctttgccagaga cgccaatgcttcttggttttaatgctcctgctacatcggaaccgagctcgttcgaatttccaccgcctccaacagaagatgaactagaaatcatcc gggaaacagcatcctcgctagattctagttttacaagaggggatttagctagtttgagaaatgctattaatcgccatagtcaaaatttctctgatttc ccaccaatcccaacagaagaagagttgaacgggagaggcggtagacca (SEQ No: 16). In another embodiment, the ActA fragment is encoded by a nucleotide molecule that comprises SEQ ID No: 16. In another embodiment, the ActA fragment is encoded by a nucleotide molecule that is a homologue of SEQ ID No: 16. In another embodiment, the ActA fragment is encoded by a nucleotide molecule that is a variant of SEQ ID No: 16. In another embodiment, the ActA fragment is encoded by a nucleotide molecule that is an isomer of SEQ ID No: 16. In another embodiment, the ActA fragment is encoded by a nucleotide molecule that is a fragment of SEQ ID No: 16. Each possibility represents a separate embodiment of the present invention.

In another embodiment, a recombinant nucleotide of the present invention comprises any other sequence that encodes a fragment of an ActA protein. Each possibility represents a separate embodiment of the present invention.

In another embodiment, the ActA fragment is any other ActA fragment known in the art. Each possibility represents a separate embodiment of the present invention.

In another embodiment of methods and compositions of the present invention, a PEST-like AA sequence is fused to the MAGE-b peptide. In another embodiment, the PEST-like AA sequence has a sequence selected from SEQ ID NO: 2-7 and 20. In another embodiment, the PEST-like sequence is any other PEST-like sequence known in the art. Each possibility represents a separate embodiment of the present invention.

In another embodiment, the PEST-like AA sequence is KENSISSMAPPASPPASPKTPIEKKHADEIDK (SEQ ID NO: 1). In another embodiment, the PEST-like sequence is KENSISSMAPPASPPASPK (SEQ ID No: 21). In another embodiment, fusion of a MAGE-b peptide to any LLO sequence that includes the 1 of the PEST-like AA sequences enumerated herein is efficacious for enhancing cell-mediated immunity against MAGE-b.

The present invention also provides methods for enhancing cell mediated and anti-tumor immunity and compositions with enhanced immunogenicity which comprise a PEST-like amino acid sequence derived from a prokaryotic organism fused to a MAGE-b antigen. In another embodiment, the PEST-like sequence is embedded within an antigen. In another embodiment, the PEST-like sequence is fused to either the amino terminus of the antigen. In another embodiment, the PEST-like sequence is fused to the carboxy terminus. As demonstrated herein, fusion of an antigen to the PEST-like sequence of LM enhanced cell mediated and anti-tumor immunity of the antigen. Thus, fusion of an antigen to other PEST-like sequences derived from other prokaryotic organisms will also enhance immunogenicity of MAGE-b. PEST-like sequence of other prokaryotic organism can be identified routinely in accordance with methods such as described by, for example Rechsteiner and Rogers (1996, Trends Biochem. Sci. 21:267-271) for LM. In another embodiment, PEST-like AA sequences from other prokaryotic organisms are identified based by this method. In another embodiment, the PEST-like AA sequence is from another Listeria species. For example, the LM protein ActA contains 4 such sequences.

In another embodiment, the PEST-like AA sequence is a PEST-like sequence from a Listeria ActA protein. In another embodiment, the PEST-like sequence is KTEEQPSEVNTGPR (SEQ ID NO: 2), KASVTDTSEGDLDSSMQSADESTPQPLK (SEQ ID NO: 3), KNEEVNASDFPPPPTDEELR (SEQ ID NO: 4), or RGGIPTSEEFSSLNSGDFTDDENSETTEEEIDR (SEQ ID NO: 5). In another embodiment, the PEST-like sequence is from Listeria seeligeri cytolysin, encoded by the Iso gene. In another embodiment, the PEST-like sequence is RSEVTISPAETPESPPATP (SEQ ID NO: 20). In another embodiment, the PEST-like sequence is from Streptolysin 0 protein of Streptococcus sp. In another embodiment, the PEST-like sequence is from Streptococcus pyogenes Streptolysin 0, e.g. KQNTASTET™ NEQPK (SEQ ID NO: 6) at AA 35-51. In another embodiment, the PEST-like sequence is from Streptococcus equisimilis Streptolysin O, e.g. KQNTANTETTTTNEQPK (SEQ ID NO: 7) at AA 38-54. In another embodiment, the PEST-like sequence has a sequence selected from SEQ ID NO: 1-7 and 20-21. In another embodiment, the PEST-like sequence has a sequence selected from SEQ ID NO: 2-7 and 20. In another embodiment, the PEST-like sequence is another PEST-like AA sequence derived from a prokaryotic organism.

PEST-like sequences of other prokaryotic organism are identified, in another embodiment, in accordance with methods such as described by, for example Rechsteiner and Rogers (1996, Trends Biochem. Sci. 21:267-271) for LM. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising both the MAGE-b peptide and the PEST-like amino acid sequence either linked at one end of the MAGE-b peptide or embedded within the MAGE-b peptide.

In another embodiment, the PEST-like sequence is identified using the PEST-find program. In another embodiment, a PEST-like sequence is defined as a hydrophilic stretch of at least 12 AA in length with a high local concentration of proline (P), aspartate (D), glutamate (E), serine (S), and/or threonine (T) residues. In another embodiment, a PEST-like sequence contains no positively charged AA, namely arginine (R), histidine (H) and lysine (K).

In another embodiment, identification of PEST motifs is achieved by an initial scan for positively charged AA R, H, and K within the specified protein sequence. All AA between the positively charged flanks are counted and only those motifs are considered further, which contain a number of AA equal to or higher than the window-size parameter. In another embodiment, a PEST-like sequence must contain at least 1 P, 1 D or E, and at least 1 S or T.

In another embodiment, the quality of a PEST motif is refined by means of a scoring parameter based on the local enrichment of critical AA as well as the motif's hydrophobicity. Enrichment of D, E, P, S and T is expressed in mass percent (w/w) and corrected for 1 equivalent of D or E, 1 of P and 1 of S or T. In another embodiment, calculation of hydrophobicity follows in principle the method of J. Kyte and R. F. Doolittle (Kyte, J and Dootlittle, R F. J. Mol. Biol. 157, 105 (1982). For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine.
Hydropathy index=10*Kyte-Doolittle hydropathy index+45

In another embodiment, a potential PEST motif's hydrophobicity is calculated as the sum over the products of mole percent and hydrophobicity index for each AA species. The desired PEST score is obtained as combination of local enrichment term and hydrophobicity term as expressed by the following equation:
PEST score=0.55*DEPST−0.5*hydrophobicity index.

In another embodiment, “PEST-like sequence” or “PEST-like sequence peptide” refers to a peptide having a score of at least +5, using the above algorithm. In another embodiment, the term refers to a peptide having a score of at least 6. In another embodiment, the peptide has a score of at least 7. In another embodiment, the score is at least 8. In another embodiment, the score is at least 9. In another embodiment, the score is at least 10. In another embodiment, the score is at least 11. In another embodiment, the score is at least 12. In another embodiment, the score is at least 13. In another embodiment, the score is at least 14. In another embodiment, the score is at least 15. In another embodiment, the score is at least 16. In another embodiment, the score is at least 17. In another embodiment, the score is at least 18. In another embodiment, the score is at least 19. In another embodiment, the score is at least 20. In another embodiment, the score is at least 21. In another embodiment, the score is at least 22. In another embodiment, the score is at least 22. In another embodiment, the score is at least 24. In another embodiment, the score is at least 24. In another embodiment, the score is at least 25. In another embodiment, the score is at least 26. In another embodiment, the score is at least 27. In another embodiment, the score is at least 28. In another embodiment, the score is at least 29. In another embodiment, the score is at least 30. In another embodiment, the score is at least 32. In another embodiment, the score is at least 35. In another embodiment, the score is at least 38. In another embodiment, the score is at least 40. In another embodiment, the score is at least 45. Each possibility represents a separate embodiment of the present invention.

In another embodiment, the PEST-like sequence is identified using any other method or algorithm known in the art, e.g. the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each stretch of appropriate length (e.g. a 30-35 AA stretch) by assigning a value of 1 to the AA Ser, Thr, Pro, Glu, Asp, Asn, or Gln. The coefficient value (CV) for each of the PEST residue is 1 and for each of the other AA (non-PEST) is 0.

Each method for identifying a PEST-like sequence represents a separate embodiment of the present invention.

“Fusion to a PEST-like sequence” refers, in another embodiment, to fusion to a protein fragment comprising a PEST-like sequence. In another embodiment, the term includes cases wherein the protein fragment comprises surrounding sequence other than the PEST-like sequence. In another embodiment, the protein fragment consists of the PEST-like sequence. Each possibility represents a separate embodiment of the present invention.

As provided herein, recombinant Listeria strains expressing PEST-like sequence-antigen fusions induce anti-tumor immunity (Example 3) and generate antigen-specific, tumor-infiltrating T cells (Example 4).

In another embodiment, “homology” refers to identity greater than 70% to a MAGE-b sequence set forth in a sequence selected from SEQ ID No: 25-43. In another embodiment, “homology” refers to identity to one of SEQ ID No: 25-43 of greater than 72%. In another embodiment, the homology is greater than 75%. In another embodiment, “homology” refers to identity to a sequence of greater than 78%. In another embodiment, the homology is greater than 80%. In another embodiment, the homology is greater than 82%. In another embodiment, “homology” refers to identity to a sequence of greater than 83%. In another embodiment, the homology is greater than 85%. In another embodiment, the homology is greater than 87%. In another embodiment, “homology” refers to identity to a sequence of greater than 88%. In another embodiment, the homology is greater than 90%. In another embodiment, the homology is greater than 92%. In another embodiment, “homology” refers to identity to a sequence of greater than 93%. In another embodiment, the homology is greater than 95%. In another embodiment, “homology” refers to identity to a sequence of greater than 96%. In another embodiment, the homology is greater than 97%. In another embodiment, the homology is greater than 98%. In another embodiment, the homology is greater than 99%. In another embodiment, “homology” refers to identity of 100% to one of SEQ ID No: 25-43. Each possibility represents a separate embodiment of the present invention.

In another embodiment, “homology” refers to identity greater than 70% to an LLO sequence set forth in a sequence selected from SEQ ID No: 17-19. In another embodiment, “homology” refers to identity to one of SEQ ID No: 17-19 of greater than 72%. In another embodiment, the homology is greater than 75%. In another embodiment, “homology” refers to identity to a sequence of greater than 78%. In another embodiment, the homology is greater than 80%. In another embodiment, the homology is greater than 82%. In another embodiment, “homology” refers to identity to a sequence of greater than 83%. In another embodiment, the homology is greater than 85%. In another embodiment, the homology is greater than 87%. In another embodiment, “homology” refers to identity to a sequence, of greater than 88%. In another embodiment, the homology is greater than 90%. In another embodiment, the homology is greater than 92%. In another embodiment, “homology” refers to identity to a sequence of greater than 93%. In another embodiment, the homology is greater than 95%. In another embodiment, “homology” refers to identity to a sequence of greater than 96%. In another embodiment, the homology is greater than 97%. In another embodiment, the homology is greater than 98%. In another embodiment, the homology is greater than 99%. In another embodiment, “homology” refers to identity of 100% to one of SEQ ID No: 17-19. Each possibility represents a separate embodiment of the present invention.

In another embodiment, “homology” refers to identity greater than 70% to an ActA sequence set forth in a sequence selected from SEQ ID No: 15-16 and 44. In another embodiment, “homology” refers to identity to one of SEQ ID No: 15-16 and 44 of greater than 72%. In another embodiment, the homology is greater than 75%. In another embodiment, “homology” refers to identity to a sequence of greater than 78%. In another embodiment, the homology is greater than 80%. In another embodiment, the homology is greater than 82%. In another embodiment, “homology” refers to identity to a sequence of greater than 83%. In another embodiment, the homology is greater than 85%. In another embodiment, the homology is greater than 87%. In another embodiment, “homology” refers to identity to a sequence of greater than 88%. In another embodiment, the homology is greater than 90%. In another embodiment, the homology is greater than 92%. In another embodiment, “homology” refers to identity to a sequence of greater than 93%. In another embodiment, the homology is greater than 95%. In another embodiment, “homology” refers to identity to a sequence of greater than 96%. In another embodiment, the homology is greater than 97%. In another embodiment, the homology is greater than 98%. In another embodiment, the homology is greater than 99%. In another embodiment, “homology” refers to identity of 100% to one of SEQ ID No: 15-16 and 44. Each possibility represents a separate embodiment of the present invention.

In another embodiment, “homology” refers to identity greater than 70% to a PEST-like sequence set forth in a sequence selected from SEQ ID No: 1-7 and 20-21. In another embodiment, “homology” refers to identity to one of SEQ ID No: 1-7 and 20-21 of greater than 72%. In another embodiment, the homology is greater than 75%. In another embodiment, “homology” refers to identity to a sequence of greater than 78%. In another embodiment, the homology is greater than 80%. In another embodiment, the homology is greater than 82%. In another embodiment, “homology” refers to identity to a sequence of greater than 83%. In another embodiment, the homology is greater than 85%. In another embodiment, the homology is greater than 87%. In another embodiment, “homology” refers to identity to a sequence of greater than 88%. In another embodiment, the homology is greater than 90%. In another embodiment, the homology is greater than 92%. In another embodiment, “homology” refers to identity to a sequence of greater than 93%. In another embodiment, the homology is greater than 95%. In another embodiment, “homology” refers to identity to a sequence of greater than 96%. In another embodiment, the homology is greater than 97%. In another embodiment, the homology is greater than 98%. In another embodiment, the homology is greater than 99%. In another embodiment, “homology” refers to identity of 100% to one of SEQ ID No: 1-7 and 20-21. Each possibility represents a separate embodiment of the present invention.

In another embodiment of the present invention, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

In another embodiment, the ActA or LLO fragment is attached to MAGE-b peptide by chemical conjugation. In another embodiment, paraformaldehyde is used for the conjugation. In another embodiment, the conjugation is performed using any suitable method known in the art. Each possibility represents another embodiment of the present invention.

In another embodiment, the MAGE-b expressing tumor targeted by methods and compositions of the present invention is a breast cancer. In another embodiment, the cancer is a melanoma. In another embodiment, the cancer is a glioma tumor. In another embodiment, the cancer is an ovarian neoplasm. In another embodiment, the cancer is a mammary carcinoma. In another embodiment, the cancer is an ependymoma.

In another embodiment, the cancer is a melanoma. In another embodiment, the cancer is a sarcoma. In another embodiment, the cancer is a carcinoma. In another embodiment, the cancer is a lymphoma. In another embodiment, the cancer is a leukemia. In another embodiment, the cancer is mesothelioma. In another embodiment, the cancer is a glioma. In another embodiment, the cancer is a germ cell tumor. In another embodiment, the cancer is a choriocarcinoma. Each possibility represents a separate embodiment of the present invention.

In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma.

In another embodiment, the cancer is an acute myelogenous leukemia (AML). In another embodiment, the cancer is a myelodysplastic syndrome (MDS). In another embodiment, the cancer is a non-small cell lung cancer (NSCLC). In another embodiment, the cancer is a Wilms' tumor. In another embodiment, the cancer is a leukemia. In another embodiment, the cancer is a lymphoma. In another embodiment, the cancer is a desmoplastic small round cell tumor. In another embodiment, the cancer is a mesothelioma (e.g. malignant mesothelioma). In another embodiment, the cancer is a gastric cancer. In another embodiment, the cancer is a colon cancer. In another embodiment, the cancer is a lung cancer. In another embodiment, the cancer is a breast cancer. In another embodiment, the cancer is a germ cell tumor. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma. Each possibility represents a separate embodiment of the present invention.

In another embodiment, the cancer is any other MAGE-b-expressing cancer known in the art. Each type of cancer represents a separate embodiment of the present invention.

As provided herein, enhanced cell mediated immunity was demonstrated for fusion proteins comprising an antigen and truncated LLO containing the PEST-like amino acid sequence, SEQ ID NO: 1. The ΔLLO used in some of the Examples was 416 amino acids long, as 88 residues from the carboxy terminus which is inclusive of the activation domain containing cysteine 484 were truncated. However, it is apparent from the present disclosure that other ΔLLO without the activation domain, and in particular cysteine 484, are efficacious in methods of the present invention. More particularly, it is believed that fusion of MAGE-b to any ΔLLO including the PEST-like amino acid sequence, SEQ ID NO: 1, can enhance cell-mediated and anti-tumor immunity elicited by the resulting vaccine.

As provided herein, fusion of an antigen to a non-hemolytic truncated form of listeriolysin O (LLO) enhanced immunogenicity. An LM vector that expresses and secretes a fusion product of Human Papilloma Virus (HPV) strain 16 E7 and listeriolysin was a more potent cancer immunotherapeutic for HPV-immortalized tumors than LM secreting the E7 protein alone. Further, a recombinant vaccinia virus that carries the gene for the fusion protein LLO-E7 is a more potent cancer immunotherapeutic for HPV-immortalized tumors than an isogenic strain of vaccinia that carries the gene for E7 protein alone. In comparison, a short fusion protein Lm-AZ/-E7 comprising the E7 antigen fused to the promoter, signal sequence and the first 7 AA residues of LLO was an ineffective anti-tumor immunotherapeutic. This short fusion protein terminates directly before the PEST-like sequence and does not contain it.

In another embodiment, the present invention provides a MAGE-b peptide fused to a truncated ActA protein, truncated LLO protein, or PEST-like sequence. As demonstrated by the data disclosed herein, an antigen fused to a truncated ActA protein, truncated LLO protein, or PEST-like sequence, when administered to an animal, results in clearing of existing tumors and the induction of antigen-specific CD8⁺ cells capable of infiltrating infected or tumor cells. Therefore, truncated ActA proteins, truncated LLO proteins, and PEST-like sequences are efficacious for enhancing the immunogenicity of MAGE-b.

“Fusion protein” refers, in another embodiment, to a protein comprising 2 or more proteins linked together by peptide bonds or other chemical bonds. In another embodiment, the proteins are linked together directly by a peptide or other chemical bond. In another embodiment, the proteins are linked together with one or more amino acids (e.g. a “spacer”) between the two or more proteins. Each possibility represents a separate embodiment of the present invention.

Fusion proteins comprising a MAGE-b peptide are, in another embodiment, prepared by any suitable method. In another embodiment, a fusion protein is prepared by cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the MAGE-b peptide is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the 2 fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The MAGE-b peptide-encoding gene is then ligated into a plasmid.

In another embodiment, the MAGE-b peptide is conjugated to the truncated ActA protein, truncated LLO protein, or PEST-like sequence by any of a number of means well known to those of skill in the art. In another embodiment, the MAGE-b peptide is conjugated, either directly or through a linker (spacer), to the ActA protein or LLO protein. In another embodiment, wherein both the MAGE-b peptide and the ActA protein or LLO protein are polypeptides, the chimeric molecule is recombinantly expressed as a single-chain fusion protein.

In another embodiment, wherein the MAGE-b peptide and/or the ActA protein, LLO protein, or PEST-like sequence is relatively short (i.e., less than about 50 amino acids) they are synthesized using standard chemical peptide synthesis techniques. Where both molecules are relatively short, in another embodiment, the chimeric molecule is synthesized as a single contiguous polypeptide. In another embodiment, the MAGE-b peptide and the ActA protein, LLO protein, or PEST-like sequence are synthesized separately and then fused by condensation of the amino terminus of one molecule with the carboxyl terminus of the other molecule thereby forming a peptide bond. In another embodiment, the MAGE-b peptide and the ActA protein, LLO protein, or PEST-like sequence are each condensed with one end of a peptide spacer molecule, thereby forming a contiguous fusion protein.

In another embodiment, the peptides and proteins of the present invention are readily prepared by standard, well-established solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky (The Practice of Peptide Synthesis, 1984, Springer-Verlag, New York). At the outset, a suitably protected amino acid residue is attached through its carboxyl group to a derivatized, insoluble polymeric. support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin coupled peptide can be treated with 20% acetic anhydride in acetonitrile. The N-blocked peptide product can then be cleaved from the resin, deprotected and subsequently isolated.

In another embodiment, to ensure that the peptide obtained from either chemical or biological synthetic techniques is the desired peptide, analysis of the peptide composition is conducted. In another embodiment, amino acid composition analysis is conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequencers, which sequentially degrade the peptide and identify the amino acids in order, may also be used to determine definitely the sequence of the peptide.

In another embodiment, prior to its use, the peptide is purified to remove contaminants. In this regard, it will be appreciated that the peptide will be purified so as to meet the standards set out by the appropriate regulatory agencies and guidelines. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Solid phase synthesis in which the C-terminal AA of the sequence is attached to an insoluble support followed by sequential addition of the remaining amino acids in the sequence is used, in another embodiment, for the chemical synthesis of the polypeptides of this invention. Techniques for solid phase synthesis are described by Barany and Merrifield in Solid-Phase Peptide Synthesis; pp. 3-284 in The Peptides: Analysis, Synthesis, Biology. Vol. 2: Special Methods in Peptide Synthesis, Part A., Merrifield, et al. J. Am. Chem. Soc., 85: 2149-2156 (1963), and Stewart et al., Solid Phase Peptide Synthesis, 2nd ed. Pierce Chem. Co., Rockford, Ill. (1984).

In another embodiment, peptides of the present invention can incorporate AA residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

In another embodiment, blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkyl amino groups such as methyl amino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

In another embodiment, other modifications are incorporated without adversely affecting the activity. In another embodiment, such modifications include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

In another embodiment, acid addition salts peptides of the present invention are utilized as functional equivalents thereof. In another embodiment, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

In another embodiment, modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

In another embodiment polypeptides are modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

In another embodiment, the chimeric fusion proteins of the present invention are synthesized using recombinant DNA methodology. Generally this involves creating a DNA sequence that encodes the fusion protein, placing the DNA in an expression cassette, such as the plasmid of the present invention, under the control of a particular promoter/regulatory element, and expressing the protein.

DNA encoding a fusion protein of the present invention are prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 1859-1862); and the solid support method of U.S. Pat. No. 4,458,066.

Chemical synthesis produces a single stranded oligonucleotide. This is converted, in another embodiment, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences may be obtained by the ligation of shorter sequences.

In another embodiment, “isolated nucleic acid” includes an RNA or a DNA sequence encoding an fusion protein of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Such modifications are detailed elsewhere herein. Any and all combinations of modifications of the nucleotide sequences are contemplated in the present invention.

In another embodiment, the present invention provides an isolated nucleic acid encoding a MAGE-b peptide operably linked to a non-hemolytic LLO, truncated ActA protein, or PEST-like sequence, wherein the isolated nucleic acid further comprises a promoter/regulatory sequence, such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

In another embodiment, a nucleotide of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaz promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto.

Expressing a MAGE-b peptide operably linked to a non-hemolytic LLO, truncated ActA protein, or PEST-like sequence using a vector allows the isolation of large amounts of recombinantly produced protein. It is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

In another embodiment, the present invention provides a vector comprising an isolated nucleic acid encoding a MAGE-b peptide operably linked to a non-hemolytic LLO, truncated ActA protein, or PEST-like sequence. The incorporation of a desired nucleic acid into a vector and the choice of vectors is well-known in the art as described in, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

In another embodiment, the present invention provides cells, viruses, proviruses, and the like, containing such vectors. Methods for producing cells comprising vectors and/or exogenous nucleic acids are well-known in the art. See, for example, Sambrook et al. (1989, Molecular Cloning: A Laboratory Manual, Cold Spring Harbor Laboratory, New York), and in Ausubel et al. (1997, Current Protocols in Molecular Biology, John Wiley & Sons, New York).

In another embodiment, the nucleic acids encoding a MAGE-b peptide operably linked to a non-hemolytic LLO, truncated ActA protein, or PEST-like sequence are cloned into a plasmid vector. In another embodiment, a recombinant Listeria strain is transfected with the plasmid vector. Each possibility represents a separate embodiment of the present invention.

Once armed with the present invention, it is readily apparent to one skilled in the art that other nucleic acids encoding a MAGE-b peptide operably linked to a non-hemolytic LLO, truncated ActA protein, or PEST-like sequence can be obtained by following the procedures described herein in the experimental details section for the generation of other fusion proteins as disclosed herein (e.g., site-directed mutagenesis, frame shift mutations, and the like), and procedures that are well-known in the art or to be developed.

Methods for the generation of derivative or variant forms of fusion proteins are well known in the art, and include, inter alia, using recombinant DNA methodology well known in the art such as, for example, that described in Sambrook et al. (1989, Molecular Cloning: A Laboratory Manual, Cold Spring Harbor Laboratory Press, New York) and Ausubel et al. (1997, Current Protocols in Molecular Biology, Green & Wiley, New York), and elsewhere herein.

In another embodiment, the present invention provides a nucleic acid encoding a MAGE-b peptide operably linked to a non-hemolytic LLO, truncated ActA protein, or PEST-like sequence, wherein a nucleic acid encoding a tag polypeptide is covalently linked thereto. That is, the invention encompasses a chimeric nucleic acid wherein the nucleic acid sequence encoding a tag polypeptide is covalently linked to the nucleic acid encoding a MAGE-b peptide-containing protein. Such tag polypeptides are well known in the art and include, for instance, green fluorescent protein (GFP), myc, myc-pyruvate kinase (myc-PK), His₆, maltose biding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The present invention also provides for analogs of ActA, LLO, and PEST-like sequences of the present invention, fragments thereof, proteins, or peptides. Analogs differ, in another embodiment, from naturally occurring proteins or peptides by conservative amino acid sequence differences, by modifications which do not affect sequence, or by both.

In another embodiment, the present invention provides a MAGE-b peptide with enhanced immunogenicity. That is, as the data disclosed herein demonstrate, a MAGE-b peptide fused to a truncated ActA protein, non-hemolytic LLO protein, or PEST-like sequence, when administered to an animal, results in a clearance of existing tumors and the induction of antigen-specific cytotoxic lymphocytes capable of infiltrating tumor or infected cells. When armed with the present disclosure, and the methods and compositions disclosed herein, the skilled artisan will readily realize that the present invention in amenable to treatment and/or prevention of a multitude of diseases.

In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art. A commercially available plasmid such as pCR2.1 (Invitrogen, La Jolla, Calif.), which is a prokaryotic expression vector with an prokaryotic origin of replication and promoter/regulatory elements to facilitate expression in a prokaryotic organism.

The present invention further comprises transforming such a Listeria strain with a plasmid comprising, an isolated nucleic acid encoding a truncated ActA protein, truncated LLO protein, or PEST-like sequence, and a MAGE-b peptide. As a non-limiting example, if an LM vaccine strain comprises a deletion in the prfA gene or the actA gene, the plasmid can comprise a prfA or actA gene in order to complement the mutation, thereby restoring function to the L. monocytogenes vaccine strain. As described elsewhere herein, methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.).

The plasmid of the present invention comprises, in another embodiment, a promoter/regulatory sequence operably linked to a gene encoding a fusion protein.

Plasmids and other expression vectors useful in the present invention are described elsewhere herein, and can include such features as a promoter/regulatory sequence, an origin of replication for gram negative and/or gram positive bacteria, and an isolated nucleic acid encoding a fusion protein. Further, the isolated nucleic acid encoding a fusion protein will have its own promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of bacteriophage lambda (P_{L} and P_{R}), the trp, recA, lacZ, lacd, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann. Rev. Genet. 18:415-442). Further examples of promoter/regulatory elements contemplated in the present invention include, but are not limited to the Listerial prfA promoter (GenBank Acc. No. Y07639), the Listerial hly promoter (GenBank Acc. No. X15127), and the Listerial p60 promoter (GenBank Acc. No. AY126342), or fragments thereof.

Proper expression in a prokaryotic cell utilizes, in another embodiment, a ribosome binding site upstream of the gene-encoding sequence. Such ribosome binding sites are disclosed, for example, by Gold, L., et al (1981, Ann. Rev. Microbiol. 35:365-404).

In another embodiment, the present invention provides methods for enhancing the immunogenicity of a MAGE-b antigen via fusion of the antigen to a non-hemolytic truncated form of listeriolysin O or ΔLLO. In another embodiment, the antigen is fused to a PEST-like sequence. In another embodiment, the PEST-like amino acid sequence is SEQ ID NO: 1, of LLO. The present invention further provides methods and compositions for enhancing the immunogenicity of a MAGE-b antigen by fusing the antigen to a truncated ActA protein, truncated LLO protein, or fragment thereof. As demonstrated by the data disclosed herein, an antigen fused to an ActA protein, when administered to an animal elicits an immune response that clears existing tumors and results in the induction of antigen-specific cytotoxic lymphocytes.

In another embodiment, fusion proteins of the present invention are produced recombinantly via a plasmid which encodes either a truncated form of the LLO comprising the PEST-like amino acid sequence of L. monocytogenes or a PEST-like amino acid sequence derived from another prokaryotic organism and the antigen. In another embodiment, the antigen is chemically conjugated to the truncated form of LLO comprising the PEST-like amino acid sequence of L. monocytogenes or a PEST-like amino acid sequence derived from another prokaryotic organism. “Antigen” refers, in another embodiment, to the native MAGE-b gene or gene product or truncated versions of these that include identified T cell epitopes. In another embodiment, these fusion proteins are then incorporated into vaccines for administration to animals, preferably humans, to invoke an enhanced immune response against the antigen of the fusion protein. In one embodiment, the fusion proteins of the present invention are delivered as DNA vaccines, RNA vaccines or replicating RNA vaccines. As will be apparent to those of skill in the art upon this disclosure, vaccines comprising the fusion proteins of the present invention are particularly useful in the prevention and treatment of infectious and neoplastic diseases.

In another embodiment, a vaccine of the present invention further comprises an adjuvant. Examples of adjuvants useful in these vaccines include, but are not limited to, unmethylated CpG, quill glycosides, CFA, QS21, monophosphoryl lipid A, liposomes, and bacterial mitogens and toxins.

The present invention further comprises administering to an animal or human an effective amount of a composition comprising a vaccine of the present invention. The construction of such strains is detailed elsewhere herein. The composition comprises, among other things, a pharmaceutically acceptable carrier. In another embodiment, the composition includes a Listeria vaccine strain comprising a truncated ActA protein, truncated LLO protein, or fragment thereof, fused to a MAGE-b peptide, and a pharmaceutically acceptable carrier.

In another embodiment, the present invention provides a kit which comprises a compound, including a MAGE-b peptide fused to a truncated LLO protein, truncated ActA protein, or a PEST-like sequence and/or a Listeria vaccine strain comprising same, an applicator, and an instructional material which describes use of the compound to perform the methods of the invention. Although model kits are described below, the contents of other useful kits will be apparent to the skilled artisan in light of the present disclosure. Each of these kits is contemplated within the present invention.

In another embodiment, the present invention provides a kit for eliciting an enhanced immune response to an antigen, the kit comprising a MAGE-b peptide fused to a truncated ActA protein, truncated LLO protein, or PEST-like sequence, and a pharmaceutically acceptable carrier, said kit further comprising an applicator, and an instructional material for use thereof.

In another embodiment, the present invention provides a kit for eliciting an enhanced immune response to an antigen. The kit is used in the same manner as the methods disclosed herein for the present invention. In another embodiment, the kit is used to administer a Listeria vaccine strain comprising a MAGE-b peptide fused to a truncated ActA protein, LLO protein, or PEST-like sequence. In another embodiment, the kit comprises an applicator and an instructional material for the use of the kit. These instructions simply embody the examples provided herein.

In another embodiment, the invention includes a kit for eliciting an enhanced immune response to an antigen. The kit is used in the same manner as the methods disclosed herein for the present invention. Briefly, the kit may be used to administer an antigen fused to an ActA protein, LLO protein, or PEST-like sequence. Additionally, the kit comprises an applicator and an instructional material for the use of the kit. These instructions simply embody the examples provided herein.

### EXPERIMENTAL DETAILS SECTION

### Example 1

### LLO-Antigen Fusions Induce Anti-Tumor Immunity

### Materials and Experimental Methods (Examples 1-2)

Cell lines

The C57BL/6 syngeneic TC-1 tumor was immortalized with HPV-16 E6 and E7 and transformed with the c-Ha-ras oncogene. TC-1 expresses low levels of E6 and E7 and is highly tumorigenic. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

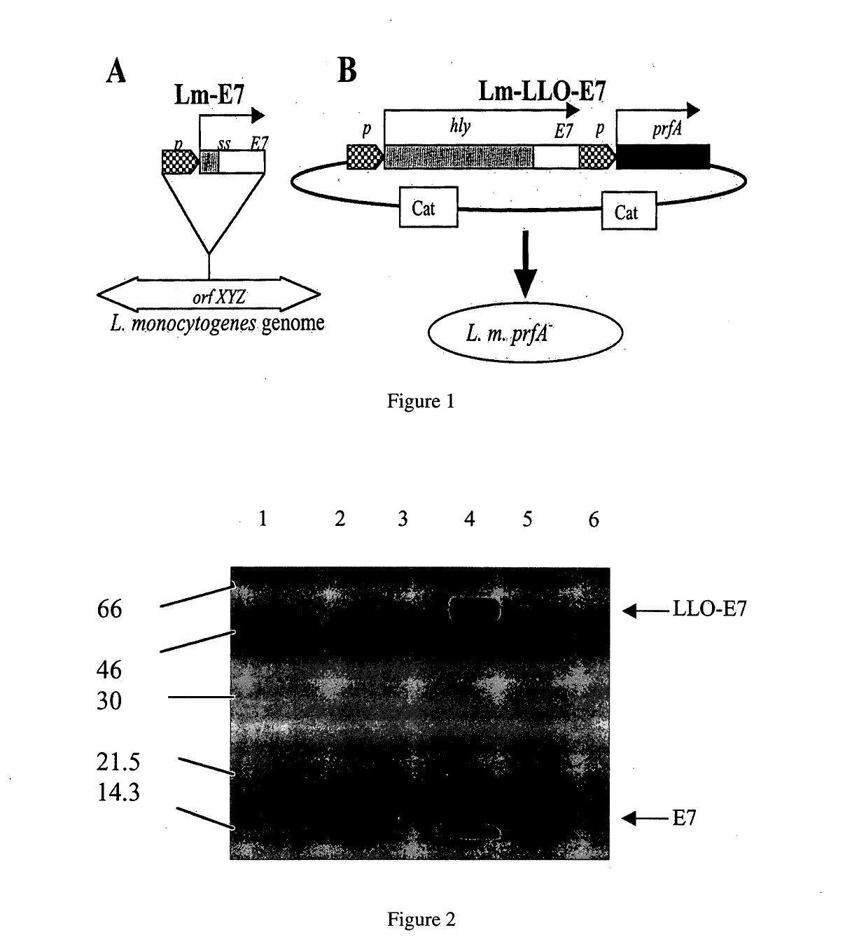

Listeria strains used were Lm-LLO-E7 (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 8; XhoI site is underlined) and 5′-GGGGACTAGTTTATGGTTTCTGAGAACA-3′ (SEQ ID No: 9; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacteriol, 165: 831, 1986), generating pGG-55. The hly promoter drives the expression of the first 441 AA of the hly gene product, counting the subsequently cleaved signal sequence (lacking the hemolytic C-terminus, referred to below as “ΔLLO,” and having the sequence set forth in SEQ ID No: 18), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 10; NheI site is underlined) and 5′-CTCCCTCGAGATCATAATTTACTTCATC-3′ (SEQ ID No: 11; XhoI site is underlined). The prfA gene was PCR amplified using primers 5′-GACTACAAGGACGATGACCGACAAGTGATAACCCGGGATCTAAATAAATCCGTTT-3′ (SEQ ID No: 12; XbaI site is underlined) and 5′-CCCGTCGACCAGCTCTTCTTGGTGAAG-3′ (SEQ ID No: 13; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 22; BamHI site is underlined) and 5′-GCTCTAGATTATGGTTTCTGAG-3′ (SEQ ID No: 23; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfz domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2)

Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of 8 mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

⁵¹Cr Release Assay

C57BL/6 mice, 6-8 wk old, were immunized i.p. with 0.1LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute−spontaneous counts per minute)/(total counts per minute−spontaneous counts per minute)]×100.

TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in counts per minute was calculated as experimental counts per minute−no Ag counts per minute.

Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7H-2 Db tetramer was performed using a FACSCalibur® flow cytometer with CellQuest® software (Becton Dickinson, Mountain View, Calif.). Splenocytes harvested 5 days after the boost were stained at room temperature (rt) with H-2 Db tetramers loaded with the E7 peptide (RAHYNIVTF) or a control (HIV-Gag) peptide. Tetramers were used at a 1/200 dilution and were provided by Dr. Larry R. Pease (Mayo Clinic, Rochester, Minn.) and by the National Institute of Allergy and Infectious Diseases Tetramer Core Facility and the National Institutes of Health AIDS Research and Reference Reagent Program. Tetramer⁺, CD8⁺, CD62L^{low} cells were analyzed.

Depletion of Specific Immune Components

CD8⁺ cells, CD4⁺ cells and IFN were depleted in TC-1-bearing mice by injecting the mice with 0.5 mg per mouse of mAb: 2.43, GK1.5, or xmg1.2, respectively, on days 6, 7, 8, 10, 12, and 14 post-tumor challenge. CD4⁺ and CD8⁺ cell populations were reduced by 99% (flow cytometric analysis). CD25⁺ cells were depleted by i.p. injection of 0.5 mg/mouse anti-CD25 mAb (PC61, provided by Andrew J. Caton) on days 4 and 6. TGF was depleted by i.p. injection of the anti-TGF-mAb (2G7, provided by H. I. Levitsky), into TC-1-bearing mice on days 6, 7, 8, 10, 12, 14, 16, 18, and 20. Mice were treated with 10⁷ Lm-LLO-E7 or Lm-E7 on day 7 following tumor challenge.

Adoptive Transfer

Donor C57BL/6 mice were immunized and boosted 7 days later with 0.1 LD₅₀ Lm-E7 or Lm-Gag. The donor splenocytes were harvested and passed over nylon wool columns to enrich for T cells. CD8⁺ T cells were depleted in vitro by incubating with 0.1 μg 2.43 anti-CD8 mAb for 30 min at rt. The labeled cells were then treated with rabbit complement. The donor splenocytes were >60% CD4⁺ T cells (flow cytometric analysis). TC-1 tumor-bearing recipient mice were immunized with 0.1 LD₅₀ 7 days post-tumor challenge. CD4⁺-enriched donor splenocytes (10⁷) were transferred 9 days after tumor challenge to recipient mice by i.v. injection.

B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

Statistics

For comparisons of tumor diameters, mean and SD of tumor size for each group were determined, and statistical significance was determined by Student's t test. p≦0.05 was considered significant.

### Results

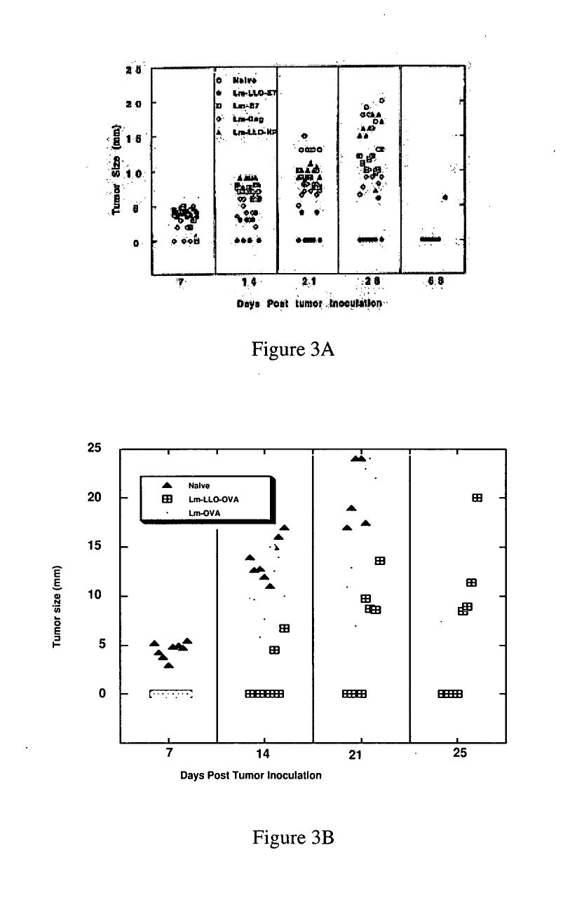

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while the other 2 mice in the group controlled their tumor growth (FIG. 3A). By contrast, immunization Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

A similar experiment was performed with the chicken ovalbumin antigen (OVA). Mice were immunized with either Lm-OVA or Lm-LLO-OVA, then challenged with either an EL-4 thymoma engineered to express OVA or the very aggressive murine melanoma cell line B16F0-Ova, which has very low MHC class I expression. In both cases, Lm-LLO-OVA, but not Lm-OVA, induced the regression of established tumors. For example, at the end of the B16F0 experiment (day 25), all the mice in the naive group and the Lm-OVA group had died. All the Lm-LLO-OVA mice were alive, and 50% of them were tumor free. (FIG. 3B).

Thus, expression of an antigen gene as a fusion protein with ΔLLO enhances the immunogenicity of the antigen.

### Example 2

### LM-LLO-E7 Treatment Elicits TC-1 Specific Splenocyte Proliferation

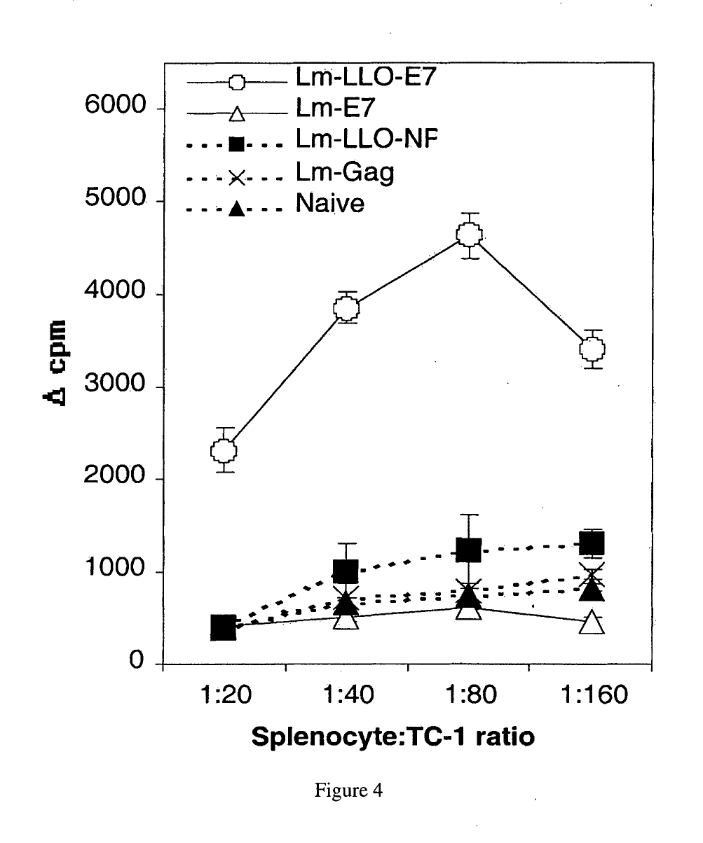

To measure induction of T cells by Lm-E7 with Lm-LLO-E7, TC-1-specific proliferative responses of splenocytes from rLm-immunized mice, a measure of antigen-specific immunocompetence, were assessed. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control immunized mice exhibited only background levels of proliferation.

### Example 3

### Fusion of NP to LLO Enhances its Immunogenicity

### Materials and Experimental Methods

Lm-LLO-NP was prepared as depicted in FIG. 1, except that influenza nucleoprotein (NP) replaced E7 as the antigen. 32 BALB/c mice were inoculated with 5×10⁵ RENCA-NP tumor cells. RENCA-NP is a renal cell carcinoma retrovirally transduced with influenza nucleoprotein NP (described in U.S. Pat. No. 5,830,702, which is incorporated herein by reference). After palpable macroscopic tumors had grown on day 10, 8 animals in each group were immunized i.p. with 0.1 LD₅₀ of the respective Listeria vector. The animals received a second immunization one week later.

### Results

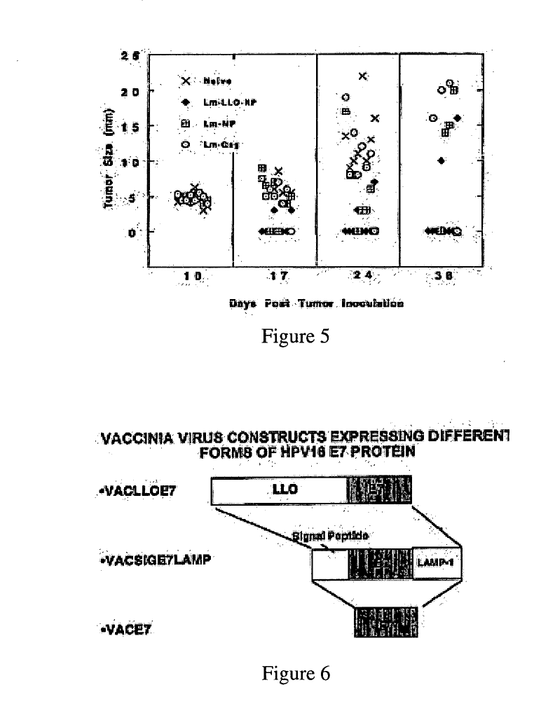

In order to confirm the generality of the finding that fusing LLO to an antigen confers enhanced immunity, Lm-LLO-NP and Lm-NP (isogenic with the Lm-E7 vectors, but expressing influenza antigen) were constructed, and the vectors were compared for ability to induce tumor regression, with Lm-Gag (isogenic with Lm-NP except for the antigen expressed) as a negative control. As depicted in FIG. 5, 6/8 of the mice that received Lm-LLO-NP were tumor free. By contrast, only 1/8 and 2/8 mice in the Lm-Gag and Lm-NP groups, respectively, were tumor free. All the mice in the naive group had large tumors or had died by day 40. Thus, LLO strains expressing NP and LLO-NP fusions are immunogenic. Similar results were achieved for Lm-LLO-E7 under different immunization protocols. Further, just a single immunization was demonstrated to cure mice of established TC-1 of 5 mm diameter.

### Example 4

### Enhancement of Immunogenicity by Fusion of an Antigen to LLO does not Require a Listeria Vector

### Materials and Experimental Methods

Construction of Vac-SigE7Lamp

The WR strain of vaccinia was used as the recipient and the fusion gene was excised from the Listerial plasmid and inserted into pSC11 under the control of the p75 promoter. This vector was chosen because it is the transfer vector used for the vaccinia constructs Vac-SigE7Lamp and Vac-E7 and would therefore allow direct comparison with Vac-LLO-E7. In this way all three vaccinia recombinants would be expressed under control of the same early/late compound promoter p7.5. In addition, SC11 allows the selection of recombinant viral plaques to TK selection and beta-galactosidase screening. FIG. 6 depicts the various vaccinia constructs used in these experiments. Vac-SigE7Lamp is a recombinant vaccinia virus that expressed the E7 protein fused between lysosomal associated membrane protein (LAMP-1) signal sequence and sequence from the cytoplasmic tail of LAMP-1. It was designed to facilitate the targeting of the antigen to the MHC class II pathway.

The following modifications were made to allow expression of the gene product by vaccinia: (a) the T5XT sequence that prevents early transcription by vaccinia was removed from the 5′ portion of the LLO-E7 sequence by PCR; and (b) an additional XmaI restriction site was introduced by PCR to allow the final insertion of LLO-E7 into SC11. Successful introduction of these changes (without loss of the original sequence that encodes for LLO-E7) was verified by sequencing. The resultant pSCl 1-E7 construct was used to transfect the TK-ve cell line CV1 that had been infected with the wild-type vaccinia strain, WR. Cell lysates obtained from this co-infection/transfection step contain vaccinia recombinants that were plaque-purified 3 times. Expression of the LLO-E7 fusion product by plaque purified vaccinia was verified by Western blot using an antibody directed against the LLO protein sequence. In addition, the ability of Vac-LLO-E7 to produce CD8⁺ T cells specific to LLOand E7 was determined using the LLO (91-99) and E7 (49-57) epitopes of Balb/c and C57/BL6 mice, respectively. Results were confirmed in a chromium release assay.

### Results

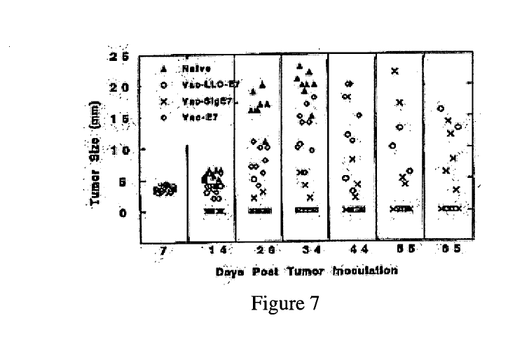

To determine whether enhancement of immunogenicity by fusion of an antigen to LLO requires a Listeria vector, a vaccinia vector expressing E7 as a fusion protein with a non-hemolytic truncated form of LLO (ΔLLO) was constructed. Tumor rejection studies were performed with TC-1 following the protocol described for Example 1. Two experiments were performed with differing delays before treatment was started. In one experiment, treatments were initiated when the tumors were about 3 mm in diameter (FIG. 7). As of day 76, 50% of the Vac-LLO-E7 treated mice were tumor free, while only 25% of the Vac-SigE7Lamp mice were tumor free. In other experiments, ΔLLO-antigen fusions were more immunogenic than E7 peptide mixed with SBAS2 or unmethylated CpG oligonucleotides in a side-by-side comparison.

These results show that (a) fusion of ΔLLO-antigen fusions are immunogenic not only in the context of Listeria, but also in other contexts; and (b) the immunogenicity of ΔLLO-antigen fusions compares favorably with other accepted vaccine approaches.

### Example 5

### ActA-E7 and PEST-E7 Fusions Confer Anti-Tumor Immunity

### Materials And Experimental Methods

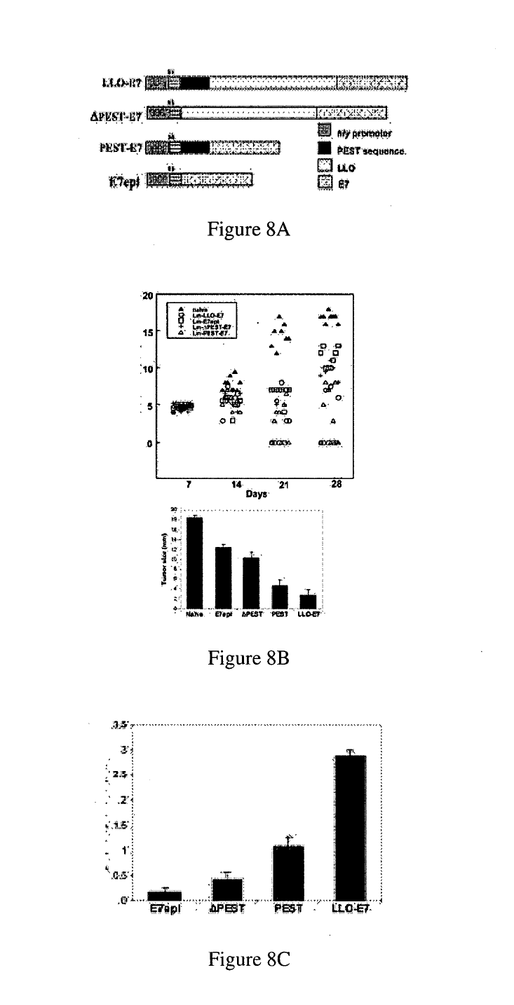

Construction of Lm-PEST-E7, Lm-ΔPEST-E7, and Lm-E7epi (FIG. 8A)

Lm-PEST-E7 is identical to Lm-LLO-E7, except that it contains only the promoter and PEST sequence of the hly gene, specifically the first 50 AA of LLO. To construct Lm-PEST-E7, the hly promoter and PEST regions were fused to the full-length E7 gene using the SOE (gene splicing by overlap extension) PCR technique. The E7 gene and the hly-PEST gene fragment were amplified from the plasmid pGG-55, which contains the first 441 AA of LLO, and spliced together by conventional PCR techniques. To create a final plasmid, pVS16.5, the hly-PEST-E7 fragment and the prfA gene were subcloned into the plasmid pAM401, which includes a chloramphenicol resistance gene for selection in vitro, and the resultant plasmid was used to transform XFL-7.

Lm-ΔPEST-E7 is a recombinant Listeria strain that is identical to Lm-LLO-E7 except that it lacks the PEST sequence. It was made essentially as described for Lm-PEST-E7, except that the episomal expression system was constructed using primers designed to remove the PEST-containing region (bp 333-387) from the hly-E7 fusion gene. Lm-E7epi is a recombinant strain that secretes E7 without the PEST region or LLO. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7epi is completely isogenic to Lm-LLO-E7, Lm-PEST-E7, and Lm-ΔPEST-E7 except for the form of the E7 antigen expressed.

### Results

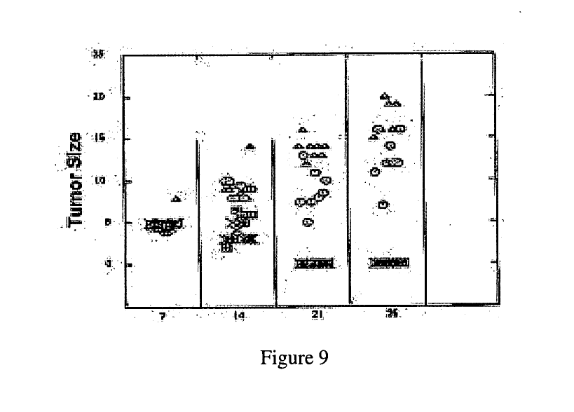

To compare the anti-tumor immunity induced by Lm-ActA-E7 versus Lm-LLO-E7, 2×10⁵ TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), (crosses) Lm-LLO-E7 (10⁸ CFU) (squares) or Lm-E7 (10⁶ CFU) (circles) on days 7 and 14. By day 26, all of the animals in the Lm-LLO-E7 and Lm-ActA-E7 were tumor free and remained so, whereas all of the naive animals (triangles) and the animals immunized with Lm-E7 grew large tumors (FIG. 9). Thus, vaccination with ActA-E7 fusions causes tumor regression.

In addition, Lm-LLO-E7, Lm-PEST-E7, Lm-ΔPEST-E7, and Lm-E7epi were compared for their ability to cause regression of E7-expressing tumors. S.c. TC-1 tumors were established on the left flank of 40 C57BL/6 mice. After tumors had reached 4-5 mm, mice were divided into 5 groups of 8 mice. Each groups was treated with 1 of 4 recombinant LM vaccines, and 1 group was left untreated. Lm-LLO-E7 and Lm-PEST-E7 induced regression of established tumors in 5/8 and 3/8 cases, respectively. There was no statistical difference between the average tumor size of mice treated with Lm-PEST-E7 or Lm-LLO-E7 at any time point. However, the vaccines that expressed E7 without the PEST sequences, Lm-ΔPEST-E7 and Lm-E7epi, failed to cause tumor regression in all mice except one (FIG. 8B, top panel). This was representative of 2 experiments, wherein a statistically significant difference in mean tumor sizes at day 28 was observed between tumors treated with Lm-LLO-E7 or Lm-PEST-E7 and those treated with Lm-E7epi or Lm-PEST-E7; P<0.001, Student's t test; FIG. 8B, bottom panel). In addition, increased percentages of tetramer-positive splenocytes were seen reproducibly over 3 experiments in the spleens of mice vaccinated with ΔPEST-containing vaccines (FIG. 8C). Thus, vaccination with ΔPEST-E7 fusions causes tumor regression.

### Example 6

### Fusion of E7 to LLO, ActA, or a Pest-Like Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated.

For tetramer staining, H-2 D^{b} tetramer was loaded with phycoerythrin (PE)-conjugated E7 peptide (RAHYNIVTF, SEQ ID NO: 24), stained at rt for 1 hour, and stained with anti-allophycocyanin (APC) conjugated MEL-14 (CD62L) and FITC-conjugated CD8β at 4° C. for 30 min. Cells were analyzed comparing tetramer⁺CD8⁺ CD62L^{low} cells in the spleen and in the tumor.

### Results

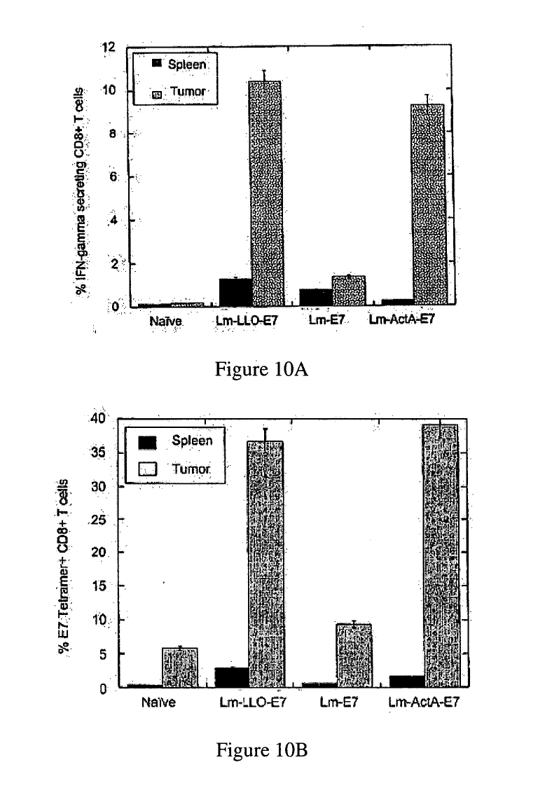

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naive). Tumors of mice from the Lm-LLO-E7 and Lm-ActA-E7 groups contained a higher percentage of IFN-gamma-secreting CD8⁺ T cells (FIG. 10A) and tetramer-specific CD8⁺ cells (FIG. 10B) than in Lm-E7 or naive mice.

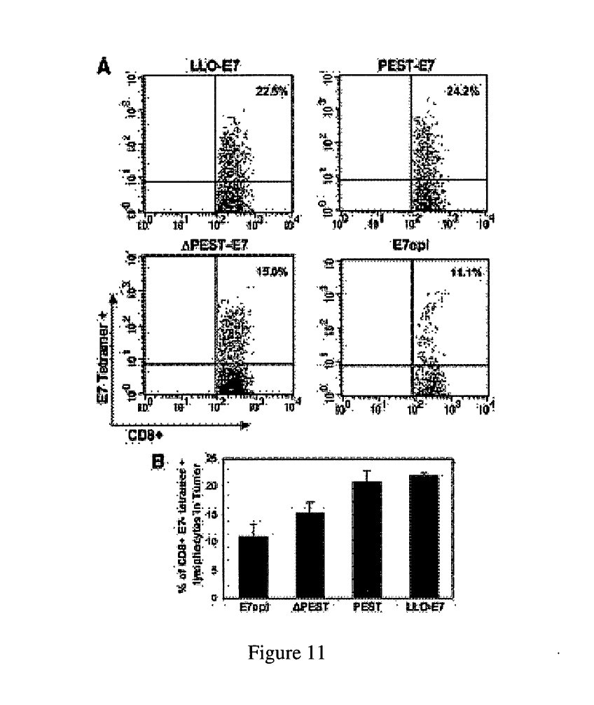

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 11A). This result was reproducible over three experiments (FIG. 11B).

Thus, Lm-LLO-E7, Lm-ActA-E7, and Lm-PEST-E7 are each efficacious at induction of tumor-infiltrating CD8⁺ T cells and tumor regression.

### Example 7

### Listeria-LLO-MAGE-b Constructs Provide Tumor Protection

### Materials and Experimental Methods

Three different fragments of the mouse Mage-b gene cDNA, namely nucleotides 3-352, 311-660, and 610-990, encoding AA 2-117, 105-220, and 204-330, respectively and the entire cDNA, were cloned as fusion proteins with Listeriolysin O (LLO) into the episomal vector pGG34 of Listeria monocytogenes (LM). The Mage-b fragments were obtained by PCR from plasmid pcDNA3.1-Mage-b/V5, whose insert has the sequence set forth in SEQ ID No: 33 and encodes the protein set forth in SEQ ID No: 32.

For each construct, a restriction site Xho1 (underlined) was included in the forward primer, and a myc Tag (italics), followed by a stop codon and restriction site XmaI (underlined) in the reverse primer. To following primers were designed:

1st fragment of Mage-b:
F^{1st}/5′:

(SEQ ID No: 45)

CTCGAGCCTAGGGGTCAAAAGAGTAAG;
and
R^{1st}/5′:

(SEQ ID No: 46)

CCCGGGTTATAGATCTTCTTCTGAAATTAGTTTTTGTTCAAACTTATCTA
GCAGGAATTC.
2nd fragment of Mage-b
F^{2nd}/5′:

(SEQ ID No: 47)

CTCGAGAGGAAGGCTAGTGTGCTGATA;
and
R^{2nd}/5′:

(SEQ ID No: 48)

CCCGGGTTATAGATCTTCTTCTGAAATTAGTTTTTGTTCTCCATGCAGAA
ATTGCCAGAC.
3rd fragment of Mage-b
F^{3rd}/5′:

(SEQ ID No: 49)

CTCGAGAACCGTGCCACTGAGCAAGAG;
and
R^{3rd}/5′:

(SEQ ID No: 14)

CCCGGGTTATAGATCTTCTTCTGAAATTAGTTTTTGTTCCATGTTAGAGG
ACTTTTGGGA.

The E7 in the pGG34 plasmid was replaced by the Mage-b fragments or complete Mage-b by digestion of the PCR fragments as well as the pGG34-E7 plasmid with XHoI and SmaI, followed by purification of the digests and ligation of pGG34 with Mage using T4 DNA polymerase (Invitrogen, Life Technologies) and transformed into E. coli. Positive colonies were analyzed by restriction digestion with XHoI and SmaI, and DNA sequencing. Subsequently, the plasmids of positive colonies were electroporated into attenuated Listeria monocytogenes (the prfA-negative Listeria monocytogenes strain, XFL-7) and analyzed for the secretion of the Mage proteins by Western blotting.

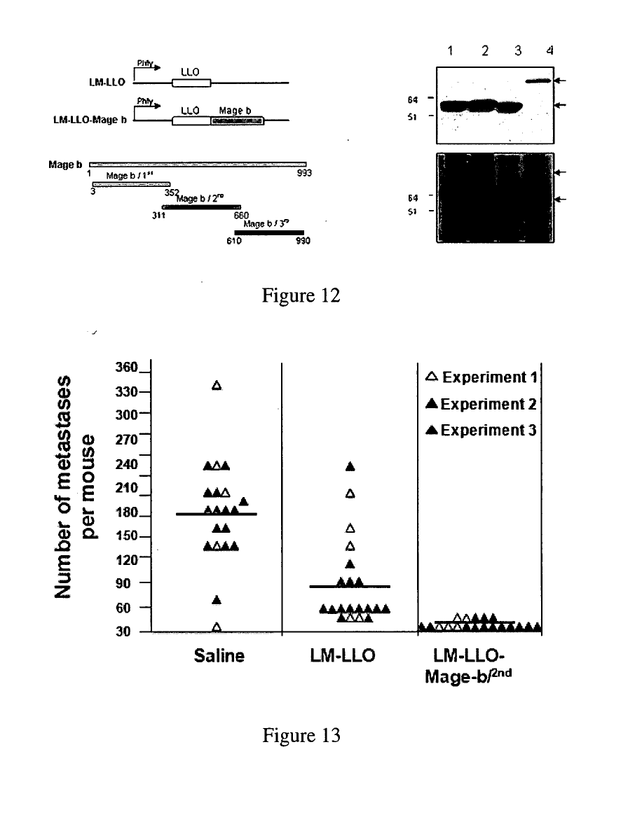

The construct containing the middle fragment, “LM-LLO-Mage-b/2^{nd},” secreted the LM-LLO-Mage-b/2nd fragment in culture medium or in the cytoplasm of cells infected with LM-LLO-Mage-b/2nd. A schematic view of the construction and characterization of the LM-LLO-Mage-b/2nd is depicted in FIG. 12.

### Results

The effect of LM-LLO-Mage-b/2nd against 4T1 breast tumor metastases in vivo was tested. To generate metastases, 105 4T1 metastatic breast tumor cells were injected into the mammary fat pad of normal BALB/c mice, resulting in 100-350 metastases in the peritoneal cavity within 2 weeks. All mice received 3 preventive vaccinations with LM-LLO-Mage-b/2nd, LM-LLO (vector control), or saline (tumor control). The number of metastases in mice injected with LM-LLO-Mage-b/2nd was reduced by 96% compared to the mice injected with saline, and by 62% compared to mice injected with the control construct missing Mage-b/2nd fragment (LM-LLO) (FIG. 13).

Thus, vaccination with Mage-b-producing LM strains and LLO-Mage-b fusions induces tumor protection.

### Example 8

### Listeria-LLO-MAGE-b Constructs Elicit Antigen-Specific Cytotoxic T Lymphocytes

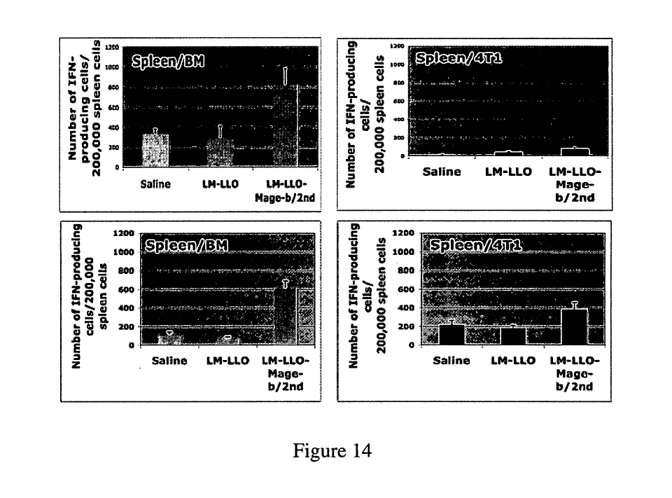

The immunogenicity of the LM-LLO-Mage-b/2nd vaccine was further tested in mice with and without 4T1 tumors. All mice received 3 preventive vaccinations. Two weeks after the last vaccination, cells from spleens of vaccinated or control mice were re-stimulated with the 4T1 tumor cell line or with autologous bone marrow cells transfected with Mage-b and GM-CSF plasmid DNA (BM/Mage-b). Three days later, the cultures were analyzed for the number of IFNγ- and IL-2-secreting cells by ELISPOT. In the spleen cultures of mice with or without tumors, a significant increase was observed in the number of IFNγ-producing cells (FIG. 14), but not IL-2-producing cells, in the LM-LLO-Mage-b/2^{nd} group compared to the control groups. This significant increase was observed after both restimulation assays, i.e., 4T1 or BM/Mage-b.

Thus, Mage-b-producing LM strains and LLO-Mage-b fusions are efficacious in the induction of antigen-specific cytotoxic T lymphocytes (CTL).

What is claimed:

1. A recombinant Listeria strain expressing a MAGE-b peptide, wherein the sequence of said MAGE-b peptide comprises a sequence selected from SEQ ID No: 34-39 or an immunogenic fragment thereof.

2. The recombinant Listeria strain of claim 1, wherein said MAGE-b polypeptide is in the form of a fusion peptide, wherein said fusion peptide further comprises a non-MAGE-b peptide, wherein said non-MAGE-b peptide enhances the immunogenicity of said fragment.

3. The recombinant Listeria strain of claim 1, wherein said non-MAGE-b peptide is a listeriolysin (LLO) peptide.

4. The recombinant Listeria strain of claim 1, wherein said non-MAGE-b peptide is selected from an ActA peptide and a PEST-like sequence peptide.

5. A vaccine comprising the recombinant Listeria strain of claim 1 and an adjuvant.

6. The recombinant Listeria strain of claim 1, wherein said recombinant Listeria strain is a recombinant Listeria monocytogenes strain.

7. The recombinant Listeria strain of claim 1, wherein said recombinant Listeria strain has been passaged through an animal host.

8. A method of inducing an anti-MAGE-b immune response in a subject, comprising administering to said subject a composition comprising the recombinant Listeria strain of claim 1, thereby inducing an anti-MAGE-b immune response in a subject.

9. A method of treating a MAGE-b expressing breast cancer in a subject, the method comprising the step of administering to said subject a composition comprising the recombinant Listeria strain of claim 1, whereby said subject mounts an immune response against said MAGE-b expressing breast cancer, thereby treating a MAGE-b expressing breast cancer in a subject.

10. A method of protecting a human subject against a MAGE-b expressing breast cancer, the method comprising the step of administering to said human subject a composition comprising the recombinant Listeria strain of claim 1, whereby said subject mounts an immune response against said MAGE-b expressing breast cancer, thereby protecting a human subject against a MAGE-b expressing breast cancer.

11. A method of treating a MAGE-b expressing breast cancer in a subject, the method comprising the step of administering to said subject a composition comprising the recombinant Listeria strain of claim 3, whereby said subject mounts an immune response against said MAGE-b expressing breast cancer, thereby treating a MAGE-b expressing breast cancer in a subject.

12. A method of protecting a human subject against a MAGE-b expressing breast cancer, the method comprising the step of administering to said human subject a composition comprising the recombinant Listeria strain of claim 3, whereby said subject mounts an immune response against said MAGE-b expressing breast cancer, thereby protecting a human subject against a MAGE-b expressing breast cancer.

13. A method of treating a MAGE-b expressing breast cancer in a subject, the method comprising the step of administering to said subject a composition comprising the recombinant Listeria strain of claim 4, whereby said subject mounts an immune response against said MAGE-b expressing breast cancer, thereby treating a MAGE-b expressing breast cancer in a subject.

14. A method of protecting a human subject against a MAGE-b expressing breast cancer, the method comprising the step of administering to said human subject a composition comprising the recombinant Listeria strain of claim 4, whereby said subject mounts an immune response against said MAGE-b expressing breast cancer, thereby protecting a human subject against a MAGE-b expressing breast cancer.

15. A recombinant polypeptide comprising a MAGE-b peptide operatively linked to a non-MAGE-b peptide, wherein said non-MAGE-b peptide is selected from a listeriolysin (LLO) peptide, an ActA peptide, and a PEST-like amino acid sequence.

16. The recombinant polypeptide of claim 15, made by a process comprising the step of translation of a nucleotide molecule encoding said recombinant polypeptide.

17. The recombinant polypeptide of claim 15, made by a process comprising the step of chemically conjugating a polypeptide comprising said MAGE-b peptide to a polypeptide comprising said non-MAGE-b peptide.

18. The recombinant polypeptide of claim 15, wherein said MAGE-b peptide is 200-400 amino acids in length.

19. A vaccine comprising the recombinant polypeptide of claim 15 and an adjuvant.

20. A recombinant vaccine vector encoding the recombinant polypeptide of claim 15.

21. A nucleotide molecule encoding the recombinant polypeptide of claim 15.

22. A vaccine comprising the nucleotide molecule of claim 21 and an adjuvant.

23. A recombinant vaccine vector comprising the nucleotide molecule of claim 21.

24. A method of inducing an anti-MAGE-b immune response in a subject, comprising administering to said subject an immunogenic composition comprising the recombinant polypeptide of claim 15, thereby inducing an anti-MAGE-b immune response in a subject.

25. A method of treating a MAGE-b expressing breast cancer in a subject, the method comprising the step of administering to said subject an immunogenic composition comprising the recombinant polypeptide of claim 15, whereby said subject mounts an immune response against said MAGE-b expressing breast cancer, thereby treating a MAGE-b expressing breast cancer in a subject.

26. A method of protecting a human subject against a MAGE-b expressing breast cancer, the method comprising the step of administering to said human subject an immunogenic composition comprising the recombinant polypeptide of claim 15, whereby said subject mounts an immune response against said MAGE-b expressing breast cancer, thereby protecting a human subject against a MAGE-b expressing breast cancer.

27. A method of inducing an anti-MAGE-b immune response in a subject, comprising administering to said subject an immunogenic composition comprising the nucleotide molecule of claim 21, thereby inducing an anti-MAGE-b immune response in a subject.

28. A method of treating a MAGE-b expressing breast cancer in a subject, the method comprising the step of administering to said subject an immunogenic composition comprising the nucleotide molecule of claim 21, whereby said subject mounts an immune response against said MAGE-b expressing breast cancer, thereby treating a MAGE-b expressing breast cancer in a subject.

29. A method of protecting a human subject against a MAGE-b expressing breast cancer, the method comprising the step of administering to said human subject an immunogenic composition comprising the nucleotide molecule of claim 21, whereby said subject mounts an immune response against said MAGE-b expressing breast cancer, thereby protecting a human subject against a MAGE-b expressing breast cancer.

30. A recombinant polypeptide comprising a fragment of a MAGE-b protein, wherein said fragment consists of amino acids 105-220 of said MAGE-b protein

31. The recombinant polypeptide of claim 30, further comprising a non-MAGE-b peptide, wherein said non-MAGE-b peptide enhances the immunogenicity of said fragment.

32. The recombinant polypeptide of claim 31, wherein said non-MAGE-b peptide is selected from a non-hemolytic listeriolysin (LLO) peptide, an ActA peptide, and a PEST-like sequence peptide.

33. The recombinant polypeptide of claim 30, made by a process comprising the step of translation of a nucleotide molecule encoding said recombinant polypeptide.

34. The recombinant polypeptide of claim 30, made by a process comprising the step of chemically conjugating a polypeptide comprising said MAGE-b peptide to a polypeptide comprising said non-MAGE-b peptide.

35. A vaccine comprising the recombinant polypeptide of claim 30 and an adjuvant.

36. A recombinant vaccine vector encoding the recombinant polypeptide of claim 30.

37. A nucleotide molecule encoding the recombinant polypeptide of claim 30.

38. A vaccine comprising the nucleotide molecule of claim 37 and an adjuvant.


SEQUENCE LISTING (50 sequences)

SEQ ID NO: 1 - protein, 32 aa, Listeria monocytogenes
KENSISSMAPPASPPASPKTPIEKKHADEIDK

SEQ ID NO: 2 - protein, 14 aa, Listeria monocytogenes
KTEEQPSEVNTGPR

SEQ ID NO: 3 - protein, 28 aa, Listeria monocytogenes
KASVTDTSEGDLDSSMQSADESTPQPLK

SEQ ID NO: 4 - protein, 20 aa, Listeria monocytogenes
KNEEVNASDFPPPPTDEELR

SEQ ID NO: 5 - protein, 33 aa, Listeria monocytogenes
RGGIPTSEEFSSLNSGDFTDDENSETTEEEIDR

SEQ ID NO: 6 - protein, 17 aa, Streptococcus pyogenes
KQNTASTETTTTNEQPK

SEQ ID NO: 7 - protein, 17 aa, Streptococcus equisimilis
KQNTANTETTTTNEQPK

SEQ ID NO: 8 - DNA, 22 bp, Artificial
  chemically synthesized
ggctcgagcatggagatacacc

SEQ ID NO: 9 - DNA, 28 bp, Artificial
  chemically synthesized
ggggactagtttatggtttctgagaaca

SEQ ID NO: 10 - DNA, 31 bp, Artificial
  chemically synthesized
gggggctagccctcctttgattagtatattc

SEQ ID NO: 11 - DNA, 28 bp, Artificial
  chemically synthesized
ctccctcgagatcataatttacttcatc

SEQ ID NO: 12 - DNA, 55 bp, Artificial
  chemically synthesized
gactacaaggacgatgaccgacaagtgataacccgggatctaaataaatccgttt

SEQ ID NO: 13 - DNA, 27 bp, Artificial
  chemically synthesized
cccgtcgaccagctcttcttggtgaag

SEQ ID NO: 14 - DNA, 60 bp, Artificial
  chemically synthesized
cccgggttatagatcttcttctgaaattagtttttgttccatgttagaggacttttggga

SEQ ID NO: 15 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 16 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 17 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 18 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 19 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 20 - protein, 19 aa, Listeria seeligeri
RSEVTISPAETPESPPATP

SEQ ID NO: 21 - protein, 19 aa, Listeria monocytogenes
KENSISSMAPPASPPASPK

SEQ ID NO: 22 - DNA, 25 bp, Artificial
  chemically synthesized
gcggatcccatggagatacacctac

SEQ ID NO: 23 - DNA, 22 bp, Artificial
  chemically synthesized
gctctagattatggtttctgag

SEQ ID NO: 24 - protein, 9 aa, Human papillomavirus type 16
RAHYNIVTF

SEQ ID NO: 25 - protein, 347 aa, Homo sapiens
MPRGQKSKLRAREKRRKAREETQGLKVAHATAAEKEECPSSSPVLGDTPTSSPAAGIPQKPQGAPPTTTAAAAVSCTESDEGAKCQGEENASFSQATTSTESSVKDPVAWEAGMLMHFILRKYKMREPIMKADMLKVVDEKYKDHFTEILNGASRRLELVFGLDLKEDNPSGHTYTLVSKLNLTNDGNLSNDWDFPRNGLLMPLLGVIFLKGNSATEEEIWKFMNVLGAYDGEEHLIYGEPRKFITQDLVQEKYLKYEQVPNSDPPRYQFLWGPRAYAETTKMKVLEFLAKMNGATPRDFPSHYEEALRDEEERAQVRSSVRARRRTTATTFRARSRAPFSRSSHPM

SEQ ID NO: 26 - DNA, 1044 bp, Homo sapiens
atgcctcggggtcagaagagtaagctccgtgctcgtgagaaacgccgcaaggcgcgagaggagacccagggtctcaaggttgctcacgccactgcagcagagaaagaggagtgcccctcctcctctcctgttttaggggatactcccacaagctcccctgctgctggcattccccagaagcctcagggagctccacccaccaccactgctgctgcagctgtgtcatgtaccgaatctgacgaaggtgccaaatgccaaggtgaggaaaatgcaagtttctcccaggccacaacatccactgagagctcagtcaaagatcctgtagcctgggaggcaggaatgctgatgcacttcattctacgtaagtataaaatgagagagcccattatgaaggcagatatgctgaaggttgttgatgaaaagtacaaggatcacttcactgagatcctcaatggagcctctcgccgcttggagctcgtctttggccttgatttgaaggaagacaaccctagtggccacacctacaccctcgtcagtaagctaaacctcaccaatgatggaaacctgagcaatgattgggactttcccaggaatgggcttctgatgcctctcctgggtgtgatcttcttaaagggcaactctgccaccgaggaagagatctggaaattcatgaatgtgttgggagcctatgatggagaggagcacttaatctatggggaaccccgtaagttcatcacccaagatctggtgcaggaaaaatatctgaagtacgagcaggtgcccaacagtgatcccccacgctatcaattcctatggggtccgagagcctatgctgaaaccaccaagatgaaagtcctcgagtttttggccaagatgaatggtgccactccccgtgacttcccatcccattatgaagaggctttgagagatgaggaagagagagcccaagtccgatccagtgttagagccaggcgtcgcactactgccacgacttttagagcgcgttctagagccccattcagcaggtcctcccaccccatgtga

SEQ ID NO: 27 - protein, 319 aa, Homo sapiens
MPRGQKSKLRAREKRRKARDETRGLNVPQVTEAEEEEAPCCSSSVSGGAASSSPAAGIPQEPQRAPTTAAAAAAGVSSTKSKKGAKSHQGEKNASSSQASTSTKSPSEDPLTRKSGSLVQFLLYKYKIKKSVTKGEMLKIVGKRFREHFPEILKKASEGLSVVFGLELNKVNPNGHTYTFIDKVDLTDEESLLSSWDFPRRKLLMPLLGVIFLNGNSATEEEIWEFLNMLGVYDGEEHSVFGEPWKLITKDLVQEKYLEYKQVPSSDPPRFQFLWGPRAYAETSKMKVLEFLAKVNGTTPCAFPTHYEEALKDEEKAGV

SEQ ID NO: 28 - protein, 346 aa, Homo sapiens
MPRGQKSTLHAREKRQQTRGQTQDHQGAQITATNKKKVSFSSPLILGATIQKKSAGRSRSALKKPQRALSTTTSVDVSYKKSYKGANSKIEKKQSFSQGLSSTVQSRTDPLIMKTNMLVQFLMEMYKMKKPIMKADMLKIVQKSHKNCFPEILKKASFNMEVVFGVDLKKVDSTKDSYVLVSKMDLPNNGTVTRGRGFPKTGLLLNLLGVIFMKGNCATEEKIWEFLNKMRIYDGKKHFIFGEPRKLITQDLVKLKYLEYRQVPNSNPARYEFLWGPRAHAETSKMKVLEFWAKVNKTVPSAFQFWYEEALRDEEERVQAAAMLNDGSSAMGRKCSKAKASSSSHA

SEQ ID NO: 29 - protein, 346 aa, Homo sapiens
MPRGQKSKLRAREKRQRTRGQTQDLKVGQPTAAEKEESPSSSSSVLRDTASSSLAFGIPQEPQREPPTTSAAAAMSCTGSDKGDESQDEENASSSQASTSTERSLKDSLTRKTKMLVQFLLYKYKMKEPTTKAEMLKIISKKYKEHFPEIFRKVSQRTELVFGLALKEVNPTTHSYILVSMLGPNDGNQSSAWTLPRNGLLMPLLSVIFLNGNCAREEEIWEFLNMLGIYDGKRHLIFGEPRKLITQDLVQEKYLEYQQVPNSDPPRYQFLWGPRAHAETSKMKVLEFLAKVNDTTPNNFPLLYEEALRDEEERAGARPRVAARRGTTAMTSAYSRATSSSSSQPM

SEQ ID NO: 30 - protein, 346 aa, Homo sapiens; identical to SEQ ID NO: 29

SEQ ID NO: 31 - DNA, 2142 bp, Homo sapiens
aggatttcatttgctcttctccaggaaccacatcacctgcccttctgcctacactcctgcctgctgtgcctaaccacagccatcatgcctcggggtcagaagagtaagctccgtgcccgtgagaaacgccagcggacccgtggtcagacccaggatctcaaggttggtcagcctactgcagcagagaaagaagagtctccttcctcttcctcatctgttttgagggatactgcctccagctcccttgcttttggcattccccaggagcctcagagagagccacccaccacctctgctgctgcagctatgtcatgcactggatctgataaaggcgacgagagccaagatgaggaaaatgcaagttcctcccaggcctcaacatccactgagagatcactcaaagattctctaaccaggaagacgaagatgttagtgcagttcctgctgtacaagtataaaatgaaagagcccactacaaaggcagaaatgctgaagatcatcagcaaaaagtacaaggagcacttccctgagatcttcaggaaagtctctcagcgcacggagctggtctttggccttgccttgaaggaggtcaaccccaccactcactcctacatcctcgtcagcatgctaggccccaacgatggaaaccagagcagtgcctggacccttccaaggaatgggcttctgatgcctctactgagtgtgatcttcttaaatggcaactgtgcccgtgaagaggaaatctgggaattcctgaatatgctggggatctatgatggaaagaggcaccttatctttggggaaccccgaaagctcatcacccaagatctggtgcaggaaaaatatctggaataccagcaggtgcccaacagtgatcccccacgctatcaattcctgtggggtccaagagctcatgcagaaaccagcaagatgaaagtcctggagtttttggccaaggtgaatgacaccacccccaataacttcccactcctttatgaagaggctttgagagatgaagaagagagagctggagcccggcccagagttgcagccaggcgtggcactacagccatgactagtgcgtattccagggccacatccagtagctcttcccaacccatgtgagatctaaggcaaattgttcactttgtggttgaaagacctgctgctttctctgttcctgtgatgcatgaataactcattgatttatctctttgttgtattttccatgatgtttcttaaaatagaaagtttatttagattcagaatataaatttagaaatggcatgcatcacacatttattgctgtttatcaggttggtttagtgataataattttgtttttgaaatacaaatagaaaatcctgaaataatttttgtgatacagagcaaaataacacggcatgggagtaaggttatccttagaaatttaaaataactccacagtaaaataggtagaatctgaagatagaaagggaagaaaagtaaaagttgctttattcgtggtttgtcttactcagttcagtctttttttgctcataaatttaaaagttacatacctggtttgcttagattattcaagaatgtggaggcctgggccaaggtcaatgacagtgtctccattgtcttccctccattaagagaagactttaagagatgagggagagagagccagagacagtgttgcaactgggcctggcatgtttcagtgtggtgtccagcagtgtctcccactccttgtgaagtctgaggtatattctttacttttgattaagaaaacacttaaccttctaattaatggagagccaaaggggagttggtgggaacaccatgtataacatatttgtatgtaaaatgatttatcttttctttttcctgtttttcagtgttctttttttaaattgtagatttatttagtttcagaatctaagtttatgaatggcatgaatcactcatttattaaaatatatcaggttggagagtgagaatttttgcattatgtaaaacaatttaaaaatcttttaagtctttttctgtgatctagaacaagataatatggcattggaatatggaatttgtgaaaaggaaattaccttgcaataaagttggtgggaccaggaagtagagaaaaaaaaagtaaaa

SEQ ID NO: 32 - protein, 330 aa, Mus musculus
MPRGQKSKTRSRAKRQQSRREVPVVQPTAEEAGSSPVDQSAGSSFPGGSAPQGVKTPGSFGAGVSCTGSGIGGRNAAVLPDTKSSDGTQAGTSIQHTLKDPIMRKASVLIEFLLDKFKMKEAVTRSEMLAVVNKKYKEQFPEIPRRTSARLELVFGLELKEIDPSTHSYLLVGKLGLSTEGSLSSNWGLPRTGLLMSVLGVIFMKGNRATEQEVWQFLHGVGVYAGKKHLIFGEPEEFIRDVVRENYLEYRQVPGSDPPSYEFLWGPRAHAETTKMKVLEVLAKVNGTVPSAFPNLYQLALRDQAGGVPRRRVQGKGVHSKAPSQKSSNM

SEQ ID NO: 33 - DNA, 993 bp, Mus musculus
atgcctaggggtcaaaagagtaagacccgctcccgtgcaaaacgacagcagtcacgcagggaggttccagtagttcagcccactgcagaggaagcagggtcttctcctgttgaccagagtgctgggtccagcttccctggtggttctgctcctcagggtgtgaaaacccctggatcttttggtgcaggtgtatcctgcacaggctctggtataggtggtagaaatgctgctgtcctgcctgatacaaaaagttcagatggcacccaggcagggacttccattcagcacacactgaaagatcctatcatgaggaaggctagtgtgctgatagaattcctgctagataagtttaagatgaaagaagcagttacaaggagtgaaatgctggcagtagttaacaagaagtataaggagcaattccctgagatccccaggagaacttctgcacgcctagaattagtctttggtcttgagttgaaggaaattgatcccagcactcattcctatttgctggtaggcaaactgggtctttccactgagggaagtttgagtagtaactgggggttgcctaggacaggtctcctaatgtctgtcctaggtgtgatcttcatgaagggtaaccgtgccactgagcaagaggtctggcaatttctgcatggagtgggggtatatgctgggaagaagcacttgatctttggcgagcctgaggagtttataagagatgtagtgcgggaaaattacctggagtaccgccaggtacctggcagtgatcccccaagctatgagttcctgtggggacccagagcccatgctgaaacaactaagatgaaagtcctggaagttttagctaaagtcaatggcacagtccctagtgccttccctaatctctaccagttggctcttagagatcaggcaggaggggtgccaagaaggagagttcaaggcaagggtgttcattccaaggccccatcccaaaagtcctctaacatgtaa

SEQ ID NO: 34 - protein, 116 aa, Mus musculus
KASVLIEFLLDKFKMKEAVTRSEMLAVVNKKYKEQFPEIPRRTSARLELVFGLELKEIDPSTHSYLLVGKLGLSTEGSLSSNWGLPRTGLLMSVLGVIFMKGNRATEQEVWQFLHG

SEQ ID NO: 35 - protein, 116 aa, Homo sapiens
EAGMLMHFILRKYKMREPIMKADMLKVVDEKYKDHFTEILNGASRRLELVFGLDLKEDNPSGHTYTLVSKLNLTNDGNLSNDWDFPRNGLLMPLLGVIFLKGNSATEEEIWKFMNV

SEQ ID NO: 36 - protein, 115 aa, Homo sapiens
KTKMLVQFLLYKYKMKEPTTKAEMLKIISKKYKEHFPEIFRKVSQRTELVFGLALKEVNPTTHSYILVSMLGPNDGNQSSAWTLPRNGLLMPLLSVIFLNGNCAREEEIWEFLNM

SEQ ID NO: 37 - protein, 116 aa, Homo sapiens
KSGSLVQFLLYKYKIKKSVTKGEMLKIVGKRFREHFPEILKKASEGLSVVFGLELNKVNPNGHTYTFIDKVDLTDEESLLSSWDFPRRKLLMPLLGVIFLNGNSATEEEIWEFLNM

SEQ ID NO: 38 - protein, 116 aa, Homo sapiens
KTNMLVQFLMEMYKMKKPIMKADMLKIVQKSHKNCFPEILKKASFNMEVVFGVDLKKVDSTKDSYVLVSKMDLPNNGTVTRGRGFPKTGLLLNLLGVIFMKGNCATEEKIWEFLNK

SEQ ID NO: 39 - protein, 127 aa, Mus musculus
MKGNRATEQEVWQFLHGVGVYAGKKHLIFGEPEEFIRDVVRENYLEYRQVPGSDPPSYEFLWGPRAHAETTKMKVLEVLAKVNGTVPSAFPNLYQLALRDQAGGVPRRRVQGKGVHSKAPSQKSSNM

SEQ ID NO: 40 - protein, 117 aa, Mus musculus
MPRGQKSKTRSRAKRQQSRREVPVVQPTAEEAGSSPVDQSAGSSFPGGSAPQGVKTPGSFGAGVSCTGSGIGGRNAAVLPDTKSSDGTQAGTSIQHTLKDPIMRKASVLIEFLLDKF

SEQ ID NO: 41 - protein, 347 aa, Homo sapiens; identical to SEQ ID NO: 25

SEQ ID NO: 42 - protein, 347 aa, Homo sapiens; identical to SEQ ID NO: 25

SEQ ID NO: 43 - protein, 347 aa, Homo sapiens; identical to SEQ ID NO: 25

SEQ ID NO: 44 - protein, 100 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNN

SEQ ID NO: 45 - DNA, 27 bp, Artificial
  chemically synthesized
ctcgagcctaggggtcaaaagagtaag

SEQ ID NO: 46 - DNA, 60 bp, Artificial
  chemically synthesized
cccgggttatagatcttcttctgaaattagtttttgttcaaacttatctagcaggaattc

SEQ ID NO: 47 - DNA, 27 bp, Artificial
  chemically synthesized
ctcgagaggaaggctagtgtgctgata

SEQ ID NO: 48 - DNA, 60 bp, Artificial
  chemically synthesized
cccgggttatagatcttcttctgaaattagtttttgttctccatgcagaaattgccagac

SEQ ID NO: 49 - DNA, 27 bp, Artificial
  chemically synthesized
ctcgagaaccgtgccactgagcaagag

SEQ ID NO: 50 - protein, 116 aa, Homo sapiens; identical to SEQ ID NO: 35